(19) United States
(12) Patent Application Publication
(10) Pub. No.: US 2008/0221395 A1
(43) Pub. Date: Sep. 11, 2008

(54) SELF-ORGANIZING MAPS IN CLINICAL DIAGNOSTICS

(76) Inventors: Steven J. Potts, San Diego, CA (US); Beryl Crossley, Foothill Ranch, CA (US); Rong Chen, Fremont, CA (US); Kevin Z. Qu, Irvine, CA (US); Richard A. Bender, Dana Point, CA (US)

Correspondence Address: FOLEY & LARDNER LLP, P.O. BOX 80278, SAN DIEGO, CA, 92138-0278, US

(21) Appl. No.: 11/617,303

(22) Filed: Dec. 28, 2006

(51) Int. Cl. A61B 5/00

(52) U.S. Cl. 600/300

(57) ABSTRACT

The present invention provides methods for the diagnosis of a disease or condition in an individual. The methods employ a primary self-organizing map trained with biological marker profiles from tissues having known diseases or conditions, in combination with a secondary self-organizing map which displays a representation of a subset of the primary self-organizing map with sample data obtained from an individual in need of diagnosis. A result is prepared from the secondary SOM(s) that reveals the extent of similarity between the known diseases or conditions with the sample data set of the individual. The result can be provided to a practitioner to aid in the diagnosis of the individual.

### FIELD OF THE INVENTION

The present invention relates to computational methods of presentation and interpretation of clinical data.

### BACKGROUND OF THE INVENTION

The following description is provided solely to assist the understanding of the present invention. None of the references cited or information provided is admitted to be prior art to the present invention.

The use of biochemical assay data such as gene expression data (i.e., gene expression profiling) is rapidly expanding the diagnosis and treatment of disease. However, large quantities of data can be difficult for a human to comprehend en masse. Thus, techniques have been developed to present complex data to individuals for evaluation. For example, statistical methodologies directed at classification of disease have been described, based on gene expression data. See Tothill et al. (Cancer Res. 2005, 65:4031-4040); Ma et al. (Arch. Pathol. Lab. Med., 2006, 130:465-473); Ramaswamy et al. (Proc. Natl. Acad. Sci, USA, 2001, 98:15149-15154); Eils (U.S. Pub. Pat. Appl. No. 2004/0076984); Botstein et al. (U.S. Pub. Appl. No. 2006/0040302); Tamayo et al. (EP 1 037 158, U.S. Pub. Appl. No. 2002/0115070); Bloom et al. (Amer. J Pathology, 2004, 164:9-16); Giordano et al. (Amer. J. Pathology, 2001, 159:1231-1238). Neural network methods also have been described in the context of expansive data, including gene expression data. See Covell et al. (Molecular Cancer Therapeutics, 2003, 2:317-332); Golub et al. (U.S. Pat. No. 6,647,341); Ingber et al. (U.S. Pat. No. 6,888,543); Buckhaults et al. (Cancer Research, 2003, 63:4144-4149); Petricoin et al. (Lancet, 2002, 359:572-577); Mavroudi et al. (Bioinformatics, 2002, 18:1446-1453); Otte et al. (U.S. Pat. No. 6,321,216); Tamayo et al. U.S. Pub. Pat. Appl. No. 2002/0115070); Mori (U.S. Pub. Pat. Appl. No. 2006/0184461); Zhang (U.S. Pat. No. 6,897,875); Hsu et al. (Bioinformatics, 2003,19:2131-2140).

### SUMMARY OF THE INVENTION

The present invention provides methods for the diagnosis of a disease or condition in an individual. These methods include assessing the level of selected biological markers within a biological sample obtained from the individual, comparing the levels of these markers in the sample with the levels of these markers in tissue or body fluid from an individual having a known disease, disorder or condition, and presenting the comparison in a form suitable for medical diagnosis.

As used herein, “biological marker” refers to a biomolecule, for example nucleic acid or protein. As a non-limiting example, the present invention provides methods for determining the primary source of a metastatic carcinoma; i.e., cancer of unknown primary. The terms “cancer of unknown primary,” “CUP,” and terms of like important refer to cancers that present in one or more metastatic sites and in which the primary site is not known. The terms “primary,” “primary site,” “primary tissue type,” “primary cancer type” and terms of like import refer in the context of cancer to the original site (i.e., tissue) in which the cancer formed. The terms “metastatic site,” “secondary site,” and terms of like import refers to other parts of the body in which cancer presents but which are not the primary site. As well understood by those of ordinary skill in the art, cancers can spread from a primary site to one or more metastatic sites. Cancers are named according to origin (i.e., primary site) regardless of where in the body the cancers spread. Because knowledge of a primary site is an important factor in determining diagnosis, treatment, and prognosis (Buckhaults et al., supra), attempts (e.g., clinical tests) are often made to determine the primary site giving rise to the metastatic site. When a primary site is determined, a cancer is no longer considered a cancer of unknown primary and is renamed according to the newly discovered primary site. For example, a lung cancer that spreads to the lymph nodes, adrenal glands, and the liver is still classified as lung cancer and not as a lymphoma (i.e., cancer of the lymph nodes), adenocarcinoma (i.e., cancer of the adrenal glands), or hepatoma (i.e., cancer of the liver). In the case of CUP, a subject may present with a metastatic cancer for which the primary cancer is occult or even no longer extant. As described herein, in some embodiments the invention contemplates gene expression level data of tissues from histologically certified primary cancer types, which data have been analyzed and transformed into a representation wherein similar types of cancer appear close to one another. The term “histologically certified primary cancer types” refers to primary cancers which have been diagnosed by an oncologist, pathologist, or other specialist using methods well known in the art of cancer diagnostics. An assay (e.g., biopsy) of a metastatic cancer can be conducted, and the levels of gene expression within the metastatic cancer can be determined by methods well known in the art. The gene expression profile of the metastatic cancer can then be compared by methods provided herein with the gene expression profiles of the histologically certified primary cancer types. The comparison is presented to a medical practitioner in a form which is understandable, and which provides assistance of diagnosis and prognosis.

In a first aspect, the invention provides a method for diagnosis of a disease or condition in an individual, the method comprising: a) providing a primary self organizing map (SOM) constructed using a plurality of data sets of measurements obtained from a plurality of individuals each having a disease or condition; b) preparing a secondary SOM using a distinct labeling set, said distinct labeling set encompassing data sets of measurements of a particular disease or condition, said secondary SOM including a sample data set obtained from a sample of said individual; and c) preparing a result from the secondary SOM that reveals the extent of similarity between the data sets of measurements of the distinct labeling set and the sample data set of the individual; whereby a medical practitioner can use the result to diagnose said disease or condition. In some embodiments, the plurality of individuals providing the data sets of measurements used to construct the primary SOM represent a plurality of diseases or conditions. In some embodiments, step b) is repeated to prepare multiple secondary SOMs for different diseases or conditions

As used herein, “self-organizing map,” “SOM,” and terms of like import refer to a clustering technique, and the representation of the result thereof, which technique groups data such that similar data are generally clustered closer than are dissimilar data. The terms “nearer” “closer” and terms of like import in this context refers to literal proximity in the SOM. Minor variations in the positioning of data comprising a SOM can be tolerated without departing from the underlying description of the SOM as provided herein and in references cited herein and known to one of ordinary skill in the art. The SOM, first enunciated by Kohonen (see e.g., Kohonen, T. “Self-Organized Formation of Topologically Correct Feature Maps”, Biological Cybernetics, 1982, 43:59-69; Kohonen, T., “The Self-Organizing Map”, Proc. of the IEEE, 1985, 73-1551-1558; Kohonen, T. “The Self-Organizing Map”, Proc. of the IEEE, 1990, 78:1464-1480; Kohonen, T., Self-Organizing Maps, Springer, 1995), is a neural network model that is capable of projecting high-dimensional input data (i.e., multivariate data vectors) onto a lower-dimensional array, typically 2-dimensional. This projection produces a lower-dimensional representation that is useful in detecting and analyzing features from the higher-dimensional input space. The term “dimension” in the context of a multivariate data vector refers to the length of the data vector, such that each of the multiple variables thereof describes a unique dimension. For example, a dimension can refer to the gene expression level, optionally normalized, of a specific gene. The term “dimension” in the context of a representation (e.g., visual representation) refers to the 1-, 2-, or 3-dimensional presentations generally used to provide information to a human. Provision of such information can be interactive as for example on a computer screen, printed, or otherwise displayed. In general, a SOM includes a set of map cells represented in a 1-, 2-, or 3-dimensional space, wherein the map cells are located in an ordered array. As used herein, the term “SOM” is understood to refer to a self-organizing map data structure and or the display thereof showing clustering of the similar data.

In some embodiments of the methods provided herein, the sets of measurements representing a plurality of different diseases or conditions. In some embodiments, the data sets of measurements are obtained from a plurality of individuals, each having a known disease or condition. In some embodiments, the sample data sets obtained from a sample from an individual in need of diagnosis are gene expression levels from a test sample. In some embodiments, the data sets are protein levels. As used herein, “sample” or “test sample” refers to any liquid or solid material that can assayed for gene expression or protein concentration. In preferred embodiments, a test sample is obtained from a biological source (i.e., a “biological sample”), a tissue sample or bodily fluid from an animal, most preferably from a human. Preferred sample tissues include, but are not limited to, lesions of specific organs including skin, colon, rectum, lung, breast, ovary, prostate, stomach, or kidney.

In some embodiments the different diseases or conditions are tumors including the following types: adrenal, brain, breast, carcinoid-intestine, cervix-adeno, cervix-squamous, endometrium, gallbladder, germ-cell-ovary, gastrointestinal stromal, kidney, leiomyosarcoma, liver, lung-adeno-large cell, lung-small cell, lung-squamous, lymphoma-B cell, lymphoma-Hodgkin, lymphoma-T cell, memigioma, mesothelioma, osteosarcoma, ovary-clear, ovary-serous, pancreas, skin-basal cell, skin-melanoma, skin-squamous, small bowel, large bowel, soft tissue-liposarcoma, soft tissue-malignant fibrous histiocytoma, soft tissue-sarcoma-synovial, stomach-adeno, testis-other, testis-seminoma, thyroid-follicular-papillary, thyroid-medullary, and urinary bladder.

In some embodiments, the sets of measurements representing a plurality of different diseases or conditions include CD (i.e., cluster of differentiation) or IHC (i.e., immunohistochemistry) markers. Representative IHC markers includes without limitation carcinoembryonic antigen (CEA), CD15, CD30, alpha fetoprotein, CD117, prostate specific antigen (PSA), and the like.

Methods of assaying gene expression levels are well known in the art, and include protein and nucleic acid determination. As used herein, “nucleic acid” refers broadly to segments of a chromosome, segments or portions of DNA, cDNA, and/or RNA. Nucleic acid may be derived or obtained from an originally isolated nucleic acid containing sample from any source (e.g., isolated from, purified from, amplified from, cloned from, reverse transcribed from sample DNA or RNA).

As used herein, “target nucleic acid” or “target sequence” refers to a sequence to be amplified and/or detected. These include the original nucleic acid sequence to be amplified, its complementary second strand of the original nucleic acid sequence to be amplified, and either strand of a copy of the original sequence which is produced by the amplification reaction. Target sequences may be composed of segments of a chromosome, a complete gene with or without intergenic sequence, segments or portions a gene with or without intergenic sequence, or sequence of nucleic acids to which probes or primers are designed. Target nucleic acids may include wild type sequences, nucleic acid sequences containing mutations, deletions or duplications, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA, preferably cDNA. Target nucleic acid may be native DNA or a copy of native DNA such as by PCR (i.e., polymerase chain reaction) amplification.

As used herein, “amplification” or “amplify” as used herein means one or more methods known in the art for copying a target nucleic acid, thereby increasing the number of copies of a selected nucleic acid sequence. Amplification may be exponential or linear. A target nucleic acid may be either DNA or RNA. The sequences amplified in this manner form an “amplicon.” While the exemplary methods described hereinafter relate to amplification using PCR, numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam et al., Nucleic Acids Res. 2001 Jun. 1; 29(11):E54-E54; Hafner et al., Biotechniques 2001 April; 30(4):852-6, 858, 860 passim; Zhong et al., Biotechniques 2001 April; 30(4):852-6, 858, 860 passim.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal amplification.

As used herein, “sense strand” means the strand of double-stranded DNA (dsDNA) that includes at least a portion of a coding sequence of a functional protein. “Anti-sense strand” means the strand of dsDNA that is the reverse complement of the sense strand.

As used herein, a “forward primer” is a primer that anneals to the anti-sense strand of dsDNA. A “reverse primer” anneals to the sense-strand of dsDNA.

As used herein, “normalized” in the context of gene expression data refers to arithmetic manipulation of observed gene expression data. Such manipulation can include the subtraction of the gene expression levels of genes which do not change in the disease or condition relative to the non-diseased state (i.e., “housekeeping” gene as known in the art.) Such manipulation can further include other arithmetic operations including multiplication by a factor, addition of an offset, negation, and the like. Further normalization procedures include subtraction of the average expression level of a specific gene from each individual sample. Exemplary housekeeping genes include without limitation those listed in Table 1. As used herein, the term “locus” in the context of the identity of a biomolecule refers to the LOCUS field in an entry of the GenBank® database. GenBank® is the NIH (National Institutes of Health) genetic sequence database which includes an annotated collection of all publicly available DNA sequences (Nucleic Acids Research, 2004 32:23-6).

TABLE 1
Exemplary housekeeping genes for gene expression level determination.

| Locus | Description |
|---|---|
| NM_001101 | Homo sapiens actin, beta (ACTB), mRNA |
| NM_000034 | Homo sapiens aldolase A, fructose-bisphosphate |
| | (ALDOA), mRNA |
| NM_002046 | Homo sapiens glyceraldehyde-3-phosphate dehydrogenase |
| | (GAPD), mRNA |
| NM_000291 | Homo sapiens phosphoglycerate kinase 1 (PGK1), mRNA |
| NM_005566 | Homo sapiens lactate dehydrogenase A (LDHA), mRNA |
| NM_002954 | Homo sapiens ribosomal protein S27a (RPS27A), mRNA |
| NM_000981 | Homo sapiens ribosomal protein L19 (RPL19), mRNA |
| NM_000975 | Homo sapiens ribosomal protein L11 (RPL11), mRNA |
| NM_007363 | Homo sapiens non-POU domain containing, octamer- |
| | binding (NONO), mRNA |
| NM_004309 | Homo sapiens Rho GDP dissociation inhibitor (GDI) alpha |
| | (ARHGDIA), mRNA |
| NM_000994 | Homo sapiens ribosomal protein L32 (RPL32), mRNA |
| NM_022551 | Homo sapiens ribosomal protein S18 (RPS18), mRNA |
| NM_007355 | Homo sapiens heat shock 90 kDa protein 1, beta |
| | (HSPCB), mRNA |
| BC006091 | TSSC4, tumor suppressing subtransferable candidate 4 |
| AL137727 | TMEM55B, transmembrane protein 55B |
| BC016680 | SP2, Sp2 transcription factor |
| BC003043 | ARF5, ADP-ribosylation factor 5 |
| AF308803 | VPS33B, vacuolar protein sorting 33B |

The plurality of data sets of measurements representing a plurality of different diseases or conditions may be narrowed in number by methods well known in the art. Standard, well-known regression techniques and other mathematical modeling may be employed to identify the most appropriate set of genes for the construction of the primary SOM, and to determine the values of the coefficients of these variables. The precise set of genes that are identified and the predictive ability of the resulting model (i.e., SOM) generally may depend upon the quality of the underlying data that is used to develop the model. Such factors as the size and completeness of the data set may be significant. The selection of the relevant variables and the computation of the appropriate coefficients are well within the skill of an ordinary person skilled in the art. In some embodiments, the plurality of data sets of measurements representing a plurality of different diseases or conditions may be narrowed in number by forward or backward stepwise logistic regression, linear regression, logistic regression, or non-stepwise logistic regression, all known to one of skill in the art.

As used herein, “map cell,” “cell,” and terms of like import refer to the individual weight vectors, and the spatial representation thereof, which form a SOM in the sense that each map cell is uniquely associated with a weight vector.

As used herein, “weight vector” refers to a multivariate data vector associated with a unique map cell (i.e., each map cell is characterized by a weight vector) which represents the results of training the SOM.

As used herein, “training vector,” “training sample” and terms of like import refer to a multivariate data vector that represents a set of characteristics used for training the SOM. As used herein, “set of characteristics used for training the SOM” refers to measurable properties of tissue having a disease or condition including, without limitation, levels of gene expression or protein levels as described herein. Weight vectors and training vectors of necessity must overlap with respect to some dimensions; however, both weight vectors and training vectors may contain additional dimensions not included in the other. For example, a training vector may include (i.e., be associated with) additional entries (e.g., name, location, and the like) which are not used in training a SOM. Conversely, a weight vector may contain additional entries (e.g., display properties of the associated map cell) which have no counterpart in a training vector. In certain embodiments, map cells can be designated (i.e., highlighted by color, shaded, annotated, or otherwise distinguished) to focus attention on an individual map cell.

As used herein, “multivariate data vector” refers to a plurality of ordered data elements. Examples of multivariate data vectors include, without limitation, the expression levels of nucleic acids and proteins in a biological sample. Weight vectors and training vectors are examples of multivariate data vectors.

As used herein, “data sets of measurements representing a plurality of different diseases or conditions” and terms of like import refer to quantified levels of biological markers obtained from samples having known disease or condition. Examples of such biological markers include, without limitation, gene expression and protein levels. Examples of biological markers suitable for use with the invention include the proteins provided in Table 2 herein. “Sample data set obtained from a sample from an individual in need of diagnosis” and terms of like import refer to quantified levels of biological markers obtained from a sample from an individual in need of diagnosis, which in this context includes diseased tissue, for example a metastatic cancer site. Assessment of such biological marker data is routinely conducted by those skilled in the art employing methods including without limitation determination of levels of nucleic acid and protein. In some embodiments, gene expression data from samples having known pathology, and from an individual in need of diagnosis, form the individual dimensions of training and weight vectors.

As used herein, “ordered array of map cells” and like terms refer to the spatial arrangement of map cells forming a SOM. For example, in a 1-dimensional context, map cells can assume e.g. a regular spacing on a line. In a 2- or 3-dimension context, map cells can assume a variety of regularly spaced arrangements, for example, square or hexagonal lattices.

As used herein, “training the SOM,” “training phase,” “SOM calculation” and like terms refer to a process wherein the weight vectors of map cells of the SOM, after initialization, are changed in response to repeated input of training vectors. As used herein, “initializing a SOM” refers to the process whereby a SOM is initially populated with weight vectors prior to training the SOM with training vectors. Methods of training the SOM are well known in the art. During the training phase, the weight vectors of the map cells gradually change so as to align according to the distribution of the training vectors.

As used herein, “primary SOM” means a self-organizing map which has been trained with a set of training vectors.

As used herein, “secondary SOM” means all or part of a primary SOM which may optionally include a sample data set obtained from a sample from an individual in need of diagnosis. The term “display of all or part of a primary SOM” refers to a selective display of individual map cells in a SOM. The term “selective display,” “distinct labeling set,” and like terms refer to indicia within the SOM data structure (e.g., subject information including diagnosis, therapeutic regimens, results of therapy, age, sex, case history reference numbers, and the like) or presented with a display of the SOM (e.g., coloring or other highlighting, flashing, annotation, and the like) to distinguish individual map cells. The selection of individual map cells in a SOM can follow any of numerous types of information associated with training vectors, including without limitation, the tissue source of the training vector most similar to the weight vector characterizing a map cell, the number of training vectors which are most similar to a specific weight vector characterizing a map cell, age, sex, prognosis, the response of the disease or condition to an agent or therapeutic regimen, and other criteria well known in the art. Preferably, a secondary SOM selectively displays map cells associated with weight vectors which are most similar to training vectors derived from a single tissue type or cancer type. For example, a secondary SOM directed at colorectal cancer selectively displays map cells which are associated with training vectors derived from tissues characterized by colorectal cancer. Accordingly, in the case of colorectal cancer the distinct labeling set contemplates training vectors derived from tissues characterized as having colorectal cancer. Additionally, a secondary SOM is optionally augmented by a sample data set obtained from a sample from an individual in need of diagnosis, which means that the map cell of the secondary SOM having a weight vector which most closely matches the sample data set is distinguished by any of the indicia described above. The terms “most similar,” “most closely matches,” and terms of like import refer to the comparison of multivariate data vectors by methods well known in the art and as described herein. Preferably, similarity is calculated as the Euclidean distance between two multivariate data vectors, as described herein. In some embodiments, similarity is calculated as the Mahalanobis, Hamming, or Chebychev distance between two multivariate data vectors, as described herein.

As used herein, “preparing a result” and terms of like import in the context of a secondary SOM refer to preparation of a measure of the extent of similarity between the data sets of measurements resulting from a disease or condition and the sample data set of an individual in need of diagnosis. In preferred embodiments, the data sets of measurements result from known (e.g., histologically certified, or otherwise diagnosed) diseases or conditions. In some embodiments, the result is a display of one or more secondary SOMs showing at least a distinct labeling set and the sample data set of the individual to be diagnosed. In some embodiments, the result is a numeric probability that the unknown disease or condition is one of the known diseases or conditions represented in the data sets of measurements used to construct the primary and secondary SOMs.

Well known techniques of computer imagery can be employed to project a 3-dimensional SOM onto a 2-dimensional display (e.g., computer screen) allowing interactive manipulation (e.g., rotation, translation, and scaling) of the 2-dimension display. In certain embodiments, the SOM can be adapted to provide a variety of functionalities. For example, the display of a SOM can be adapted such that each map cell thereof is independently pickable.

As used herein, “pickable” refers to the ability of a computer displayed object to be picked (i.e., chosen, identified, highlighted, or otherwise designated) in response to the action of a computer user. In some embodiments, the user action is the positioning of a cursor by, for example, the movement of a computer pointing device (e.g., computer mouse and the like) which is optionally clicked after positioning. In some embodiments, annotation associated with a picked map cell is displayed to a computer user in response to a picking action by the user. Annotation so displayed can provide a variety of information, including without limitation selected case history data including previous therapeutic regimens and responses thereto, age, sex, and other factors known to one skilled in the art. In some embodiments of methods provided herein, information associated with a map cell of a primary or secondary SOM is displayed. In some embodiments, the information associated with a map cell is displayed after the map cell is picked. In some embodiments, the displayed information comprises annotation associated with the training vectors which correspond to the picked map cell. In some embodiments, the display further comprises annotation associated with map cells near the picked map cell. As used herein “near the picked map cell” and like terms refer to map cells in proximity (e.g., nearest neighbor, next-nearest neighbor, and the like) to a picked map cell.

As used herein, “data element,” “scalar,” and like terms refer to the individual components of a multivariate data vector, each occupying a different dimension of the multivariate data vector. Such data elements can be continuous (e.g., a real number) or discrete (e.g., on/off, yes/no, male/female, and the like).

As used herein, “clustering technique,” “method of clustering,” and like terms refer to a variety of techniques whereby data are grouped (i.e., segregated based on similarity). In some embodiments, clustering is achieved by K-means clustering, hierarchical clustering, or expectation maximization clustering. The term “representation of clustering technique” refers to a printed or otherwise displayed (e.g., computer image) representation of the result of a clustering technique. A SOM is a clustering technique and a representation of a clustering technique. Representations of clustering techniques can be 1-, 2-, or 3-dimensional, preferably 2-dimensional (e.g., printed or displayed as a computer image).

As used herein, “Euclidean distance” is used in the conventional sense to refer to the distance d_{AB} in an N-dimension space between multivariate data vectors A and B having N components aᵢ and bᵢ, respectively, according to the generalized Pythagorean Theorem, Eqn. 1:

$${d}_{AB} = \sqrt{{\sum }_{i = 1}^{N} {( {a}_{i} - {b}_{i} )}^{2}} \qquad ( 1 )$$

Thus, Euclidian distance is calculated pairwise with respect to individual ordered data elements of a pair of multivariate data vectors.

In another aspect, the invention provides a method for diagnosis of a disease or condition in an individual comprising: a) providing a primary self organizing map (SOM) constructed using a plurality of data sets of measurements representing a plurality of different diseases or conditions, wherein the primary SOM includes at least one distinct labeling set, which distinct labeling set represents a disease or condition; b) forming at least one secondary SOM using the primary SOM with a sample data set obtained from a sample from an individual, thereby providing a display of the sample data set with respect to at least one distinct labeling set, whereby a medical practitioner can diagnose a disease or condition from the display.

In another aspect, the invention provides a method for diagnosis of a disease or condition in an individual, which method includes the following steps: a) constructing a primary self organizing map (SOM) by using a plurality of data sets of measurements representing a plurality of different diseases or conditions; b) forming at least one secondary SOM by augmenting a primary SOM with a sample data set obtained from a sample from an individual in need of diagnosis, wherein such secondary SOM displays the sample data set with respect to a distinct labeling set which represents a disease or condition; and c) providing at least one secondary SOM to a medical practitioner for diagnosing a disease or condition.

In another aspect, the invention provides a method for constructing a self-organizing map useful in the diagnosis of an individual suffering from a disease or condition, the method comprising: a) constructing a primary self organizing map by using a plurality of data sets of measurements, the data sets representing a plurality of different diseases or conditions, with the data sets obtained from a plurality of individuals each having a disease or condition; and b) forming at least one secondary SOM using at least one distinct labeling set, each distinct labeling set encompassing data sets of measurements of a particular disease or condition, with the secondary SOM including a sample data set obtained from a sample of the individual suffering from a disease or condition, thereby providing a SOM suitable for diagnosis of a disease or condition in the individual.

In another aspect, the invention provides methods for constructing a SOM useful in the diagnosis of an individual suffering from a disease or condition, which include the following steps: a) constructing a primary self organizing map (SOM) by using a plurality of data sets of measurements representing a plurality of different diseases or conditions, wherein the primary SOM comprises at least one distinct labeling set, the distinct labeling set representing a disease or condition; and b) forming at least one secondary SOM using the primary SOM with a sample data set obtained from a sample from the individual, thereby providing a display of the sample data set with respect to the at least one distinct labeling set, thereby providing a SOM suitable for diagnosis of a disease or condition in said individual.

In another aspect, the invention provides methods for constructing a SOM useful in the diagnosis of an individual suffering from a disease or condition, which include the following steps: a) constructing a primary self organizing map (SOM) by using a plurality of data sets of measurements representing a plurality of different diseases or conditions; and b) forming at least one secondary SOM by augmenting the primary SOM with a sample data set obtained from a sample from the individual suffering from a disease or condition, wherein the at least one secondary SOM displays the sample data set with respect to a distinct labeling set, and wherein the distinct labeling set represents a disease or condition; thereby providing a SOM suitable for diagnosis of a disease or condition in an individual.

In another aspect, the invention provides a method of displaying a self organizing map useful in the diagnosis of an individual suffering from a disease or condition, the method comprising: a) constructing a primary self organizing map by using a plurality of data sets of measurements, the data sets representing a plurality of different diseases or conditions, with the data sets obtained from a plurality of individuals each having a disease or condition; b) forming at least one secondary SOM using at least one distinct labeling set, the distinct labeling set encompassing data sets of measurements of a particular disease or condition, and the secondary SOM including a sample data set obtained from a sample of said individual; and c) displaying said primary SOM or said at least one secondary SOM.

In another aspect, the invention provides a method for displaying a SOM useful in the diagnosis of an individual suffering from a disease or condition, which method includes the following steps: a) providing a primary self organizing map (SOM) constructed using a plurality of data sets of measurements representing a plurality of different diseases or conditions, wherein the primary SOM comprises at least one distinct labeling set, the distinct labeling set representing a disease or condition; b) forming at least one secondary SOM by using the primary SOM with a sample data set obtained from a sample from the individual, thereby providing a display of the sample data set with respect to the at least one distinct labeling set, and c) displaying the primary SOM or the at least one secondary SOM.

In another aspect, the invention provides methods for displaying a SOM useful in the diagnosis of an individual suffering from a disease or condition, wherein include the following steps: a) constructing a primary SOM by using a plurality of data sets of measurements representing a plurality of different diseases or conditions; b) forming at least one secondary SOM by augmenting the primary SOM with a sample data set obtained from a sample from the individual suffering from a disease or condition, wherein the at least one secondary SOM displays the sample data set with respect to a distinct labeling set, and wherein the distinct labeling set represents a disease or condition; and c) displaying at least one of said primary SOM or said at least one secondary SOM.

In another aspect, the invention provides a program product comprising machine-readable program code for causing a machine to perform the following method steps: a) constructing a primary self organizing map using a plurality of data sets of measurements obtained from a plurality of individuals each having a disease or condition; and b) preparing a secondary SOM using at least one distinct labeling set, the distinct labeling set encompassing data sets of measurements of a particular disease or condition, with the secondary SOM including a sample data set obtained from a sample of said individual. In some embodiments, the invention provides a program product further comprising machine-readable program code for causing a machine to perform the following method steps: c) preparing a result from the secondary SOM that reveals the extent of similarity between the data sets of measurements of the distinct labeling set and the sample data set of the individual suffering from a disease or condition. In some embodiments of methods related to program products provided herein, there is provided machine-readable code for causing a machine to display information associated with a map cell of a primary or secondary SOM. In some embodiments, the information associated with a map cell is displayed after the map cell is picked. In some embodiments, the displayed information comprises annotation associated with the training vectors which correspond to the picked map cell. In some embodiments, the display further comprises annotation associated with map cells near the picked map cell.

In another aspect, the invention provides program products which include machine-readable program code for causing a machine to perform the following method steps: a) constructing a primary self organizing map (SOM) by using a plurality of data sets of measurements representing a plurality of different diseases or conditions, wherein the primary SOM comprises at least one distinct labeling set, the distinct labeling set representing a disease or condition; and b) forming at least one secondary SOM using the primary SOM with a sample data set obtained from a sample from an individual suffering from a disease or condition, wherein said at least one secondary SOM displays said sample data set with respect to a distinct labeling set.

In another aspect, the invention provides program products which include machine-readable program code for causing a machine to construct a primary self organizing map (SOM) by using a plurality of data sets of measurements representing a plurality of different diseases or conditions, wherein the primary SOM comprises at least one distinct labeling set, the distinct labeling set representing a disease or condition.

In another aspect, the invention provides program products which include machine-readable program code for causing a machine to form at least one secondary SOM using a primary SOM with a sample data set obtained from a sample from an individual suffering from a disease or condition, wherein the at least one secondary SOM displays the sample data set with respect to a distinct labeling set.

In another aspect, the invention provides program products which include machine-readable program code for causing a machine to perform the following method steps: a) constructing a primary SOM by using a plurality of data sets of measurements representing a plurality of different diseases or conditions; and b) forming at least one secondary SOM by augmenting the primary SOM with a sample data set obtained from a sample from an individual suffering from a disease or condition, wherein the at least one secondary SOM displays the sample data set with respect to a distinct labeling set, which distinct labeling set represents a disease or condition.

In another aspect, the invention provides a method for providing therapy response information associated with at least one pickable map cell of a primary or secondary SOM, the method comprising: a) providing annotation of therapy response information for at least one pickable map cell of a primary or secondary SOM; and b) displaying the therapy response information after the map cell is picked. In some embodiments, the method further comprises displaying therapy response information of map cells near the picked map cell.

In another aspect, the invention provides a method for reducing the number of biological markers required to construct a primary SOM useful for the diagnosis of an individual having a disease or condition, the method comprising using a reduction method to find the minimum set of biological markers that contribute a model to predict the possible diseases or conditions, wherein the reduction method is selected from the group consisting of forward stepwise logistic regression, backward stepwise logistic regression, linear regression, logistic regression, and non-stepwise logistic regression, As used herein “reduction method” refers to a mathematical method of eliminating data while retaining most of the underlying information. In some embodiments, the biological markers are particular genes. In some embodiments, the biological markers are levels of particular proteins. In some embodiments, the disease or condition is cancer of unknown primary.

In another aspect, the invention provides a method for diagnosis of cancer of unknown primary in an individual, said method comprising: a) providing a primary self organizing map (SOM) constructed using a plurality of data sets of measurements obtained from a plurality of individuals representing a plurality of particular cancers; b) preparing a plurality of secondary SOMs each using a distinct labeling set, with each of the distinct labeling sets encompassing data sets of measurements obtained from individuals having a particular cancer, and with the secondary SOM including a sample data set obtained from a sample of said individual; c) preparing a result from the plurality of secondary SOMs that reveals the extent of similarity between the data sets of measurements of the distinct labeling set and the sample data set of the individual; and d) providing the result to a medical practitioner for use to diagnosis cancer of unknown primary, wherein the result is selected from the group consisting of a primary SOM, one or more secondary SOMs, a display of a primary SOM, a display of one or more secondary SOMs, and a probability that the sample data set is one or more of the particular cancers.

### BRIEF DESCRIPTION OF THE DRAWINGS

FIG. 1 provides an exemplary schematic flow of steps in the construction of a primary SOM.

FIG. 2 provides an exemplary secondary SOM. Legend: solid filled black: map cell representing sample data set from individual in need of diagnosis; clusters obtained from a clustering of training samples: diagonal stripes, horizontal stripes, and solid gray highlighting in order of Euclidean distance from the map cell representing the sample data set.

FIG. 3 is an exemplary set of secondary SOMs, suitable for presentation to a practitioner for diagnosing cancer of unknown primary. Legend: solid filled black: map cell representing sample data set from individual in need of diagnosis; other clusters obtained from a clustering of distinct labeling sets: solid filled gray, crosshatched, diagonal stripes, respectively in order of proximity to sample data set from individual in need of diagnosis.

### DETAILED DESCRIPTION OF THE INVENTION

The construction of primary SOMs as described herein employs methodologies and software tools well known to the skilled artisan. Descriptions of suitable methods of construction are provided herein and by references described herein. Software packages which provide computational support for the construction of SOMs are available as commercial and public domain software packages including, without limitation, MATLAB® (The Mathworks, Inc., Natick, Mass.) and the SOM Toolbox for MATLAB® (Laboratory of Computer and Information Science, Helsinki University of Technology, Finland).

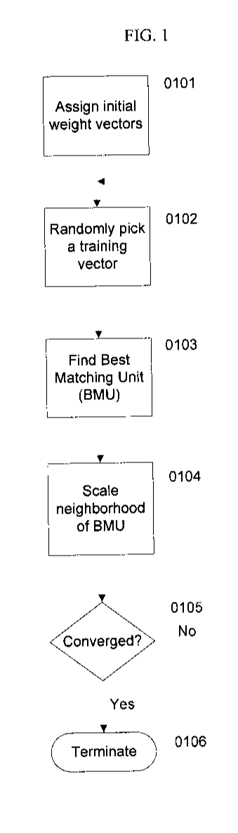

Briefly, construction of 2-dimensional SOMs may generally follow the steps as diagrammed in FIG. 1. Initially, each map cell (e.g., rectangular or hexagonal lattice point in a 2-dimension SOM) is assigned an initial weight vector (Step 0101). Many methods for the initial assignment of weight vectors are known to the skilled artisan including, without limitation, random assignment of a number to each scalar forming the weight vectors. The term “random” refers to equal probability for any of a set of possible outcomes. The numeric value of such randomly assigned scalar values may be approximately bounded at the lower and upper extrema by the corresponding extrema observed in the training vectors. Another method of initiation of weight vectors include a systematic (e.g., linear) variation in the range of each dimension of each weight vector to approximately overlap the corresponding range observed in the training vectors. In yet another method of initialization, the weights are initialized by values of the vectors ordered along a two-dimension subspace spanned by the two principal eigenvectors of the training vectors obtaining by methods of orthogonalization well known in the art (e.g., Gram-Schmidt orthogonalization). In yet a further initialization procedure, initial values are set to randomly chosen patterns of the training sample.

In step 0102, a training vector is selected. The selection may be random or systematic, preferably random. When a training vector is selected, the Euclidean distance between the selected training vector and each weight vector of the SOM is calculated.

In step 0103, the weight vector having the smallest Euclidean distance is declared the “best matching unit” (BMU). Once a BMU is identified, the neighborhood about this BMU is optionally scaled (step 0104) by methods well known in the art.

At step 0105 a decision is made whether to re-iterate processes 0102-0104, or to terminate construction of the SOM. This decision is based on whether a predefined convergence criterion has been met. The term “convergence criterion” in the context of SOM construction refers to any of a variety of metrics available to the skilled artisan. Such criteria include an absolute iteration limit (e.g., 100, 200, 500, 1000, 2000, 5000, or even more), an absolute largest change in Euclidean distance between the selected training vector and each weight vector of the SOM (e.g., 100, 10, 1, 0.1, 0.01, 0.001, and even less), a relative largest change in Euclidean distance between the selected training vector and each weight vector of the SOM (e.g., 10%, 1%, 0.1%, 0.01%, and even less), or any of these criteria additionally coupled with a requirement that all training vectors be selected a minimum number of times (e.g, 1, 2, 3, 4, 5, 10, 20, 50, 100, or even more). After convergence is reached, the procedure terminates (step 0106).

In some embodiments of methods provided herein for the diagnosis of a disease or condition in an individual, each of the plurality of diseases or conditions which are represented in data sets of measurements contemplated in the construction of a primary SOM is a cancer. As used herein “specific cancers,” “particular cancers” and terms of like import contemplated in this context include without limitation melanoma, pancreatic cancer, colorectal cancer, non-small cell lung cancer, breast cancer, small cell lung cancer, ovarian cancer, prostate cancer, stomach cancer, or kidney cancer.

In certain embodiments of methods provided herein, the sample data set obtained from a sample from an individual in need of diagnosis, and the data sets of measurements which represent a plurality of different diseases or conditions, comprise data vectors of scalars (i.e., multivariate data vectors). The scalars may be continuous or discrete, as understood by one of skill in the art. In preferred embodiments, the sample data set is isomorphic with the data sets of measurements representing a plurality of different diseases or conditions used to construct the primary and secondary SOMs. As used herein, “isomorphic” refers to correspondence of each element, on an element by element basis, of multivariate data vectors used to construct a SOM. For example without limitation, two multivariate data vectors are isomorphic if each dimension thereof used in construction of a SOM represents the same biological marker. In some embodiments, the dimensionality of the data vectors of scalars described herein is greater than 2. In some embodiments, the dimensionality of the data vectors of scalars described herein is greater than or equal to 2, 3, 4, 5, 10, 15, 20, 25, 29, 40, 50, 75, 87, 100, or even more. In some embodiments, the dimensionality of the data vectors of scalars described herein is at least 20. In some embodiments, the dimensionality of the data vectors of scalars described herein is at least 29. In some embodiments, the dimensionality of the data vectors of scalars described herein is 29.

In certain embodiments, a plurality of secondary SOMs, each employing a different distinct labeling set, are formed by methods described herein. Exemplary distinct labeling sets include without limitation distinct labeling sets directed at melanoma, pancreatic cancer, colorectal cancer, non-small cell lung cancer, breast cancer, small cell lung cancer, ovarian cancer, prostate cancer, stomach cancer, or kidney cancer.

In certain embodiments, the medical practitioner to whom the at least one secondary SOM is provided is a non-veterinary medical practitioner.

In certain embodiments, the individual in need of diagnosis presents with cancer of unknown primary. In some embodiments, diagnosis of the individual is the determination of the primary source of a metastatic cancer.

In certain embodiments, a method of diagnosis of a disease or condition in an individual further includes a step of providing to a medical practitioner a probability P_{related}ⁱ that the sample data set is related to one of the different diseases or conditions represented by the plurality of data sets of measurements.

In certain embodiments, the calculation of P_{related}ⁱ includes the following steps: i) determining a plurality of nearest neighbors of the sample data set with respect to the data sets of measurements representing a plurality of different diseases or conditions; and ii) determining if the plurality of nearest neighbors so calculated all represent the same disease or conditions. As used herein, “nearest neighbor” and terms of like import refer to the data sets of measurements representing a plurality of diseases or conditions which are most similar to the sample data set obtained from an individual in need of diagnosis. In this context, similarity may be assessed by calculation of the Euclidean distance as described herein. In some embodiments, similarity may be assessed by calculation of the Mahalanobis distance, Hamming distance, or Chebychev distance. Thus, if a rank ordering of data set of measurements were constructed using the Euclidean distance, for example without limitation, with respect to the sample data set obtained from an individual in need of diagnosis as a metric for ranking, the nearest neighbors would contiguously occupy the rank ordering with the lowest Euclidean distances. The number of nearest neighbors can be any positive integer less than or equal to the number of data sets of measurements representing a plurality of diseases or conditions, for example 2, 3, 4, 5, 6, 7, 8, 9, 10, 15, 20, or even more. Preferably, the number of nearest neighbors is 2, 3 or 4, more preferably 3.

In certain embodiments, when each of the plurality of nearest neighbors represents the same disease or condition, P_{related}ⁱ is assigned a value of 1, corresponding to 100% probability that the sample data set obtained from the individual in need of diagnosis is similar in gene expression profile to data sets obtained from tissue having the disease or condition of the nearest neighbors.

In certain embodiments, when the plurality of nearest neighbors do not each represent the same disease or condition, P_{related}ⁱ is calculated by evaluating a probability P_{cluster}ⁱ and equating P_{related}ⁱ with P_{cluster}ⁱ.

In certain embodiments, P_{cluster}ⁱ is calculated by evaluating the expression

$${P}_{cluster}^{t} = \frac{\frac{1}{{d}_{j}^{2}}}{{\sum }_{p = 1}^{T} \frac{1}{{d}_{p}^{2}}} \qquad ( 2 )$$

for one or more of the diseases or conditions represented in the plurality of nearest neighbors calculated as described herein, wherein in Eqn. (2) dⱼ is the Euclidian distance between the sample data set obtained from a sample from the individual in need of diagnosis and the closest cluster center of T clusters obtaining from a clustering of the distinct labeling sets representing the disease or condition represented in the plurality of nearest neighbors, and dₚ is the Euclidean distance between the sample data set and any of the T cluster centers.

As used herein, “clustering of the distinct labeling sets” refers to a clustering procedure wherein data sets representing the same disease or condition are clustered. For example without limitation, if the disease or condition were melanoma, then the clustering of the distinct labeling set would be over all data sets representing melanoma. Using methodology well known in the art, clustering of the distinct labeling set can be initiated for example by a hierarchical clustering, wherein the similarity, as measured by for example Euclidean distance between each pair of training samples is calculated. All samples representing a specific disease or condition are then grouped into a binary hierarchical tree using the method of simple linkage, well known in the art. The resulting hierarchical tree is then cut into clusters using an inconsistency coefficient, which as known in the art characterizes each link in a cluster tree by comparing its length with the average length of other links at the same level of hierarchy. The higher the value of the inconsistency coefficient, the less similar the objects connected by the link. The inconsistency coefficient criterion can assume any real value, preferably 1.0. After the cutting of clusters using an inconsistency coefficient, all single-sample clusters are removed. A cluster center is then defined for each remaining cluster, which cluster center has in each dimension the arithmetic mean of the corresponding dimensions of the training samples included within the cluster. Accordingly, the sum in Eqn. (2) is over all training sample clusters except single-sample clusters, with the exception that for diseases or conditions (e.g., tissues having a histologically certified cancer) which have multiple clusters, only the closest such cluster center is used in the sum of Eqn. (2).

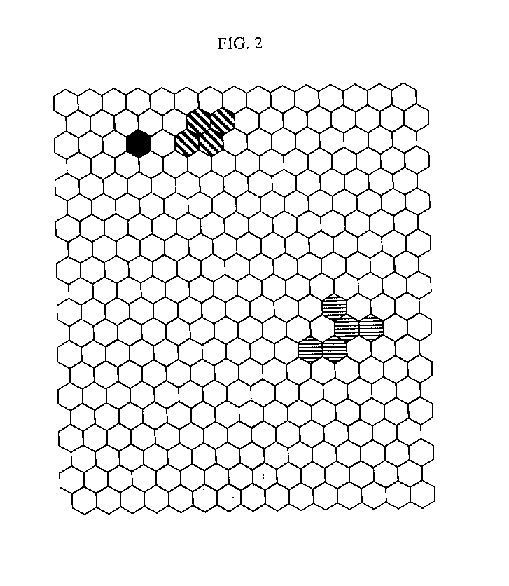

In embodiments of the invention provided herein, at least one secondary SOM displays the sample data set with respect to a distinct labeling set, wherein the distinct labeling set represents a disease or condition. An idealized secondary SOM is shown in FIG. 2. In FIG. 2, the map cell representing the sample data set obtained from a sample from an individual in need of diagnosis is displayed as a solid hexagon in the upper left corner. In this idealized figure, 17 additional map cells are highlighted which correspond to 17 different data sets of measurement arising from 17 unique training samples. These 17 training samples have been classified into 3 clusters, having diagonal stripes, horizontal stripes, and solid gray highlighting in order of Euclidean distance from the map cell representing the sample data set.

In certain embodiments, when the plurality of nearest neighbors do not each represent the same disease or condition, P_{related}ⁱ is calculated by evaluating a probability Pₜᵢₛₛᵤₑⁱ and equated P_{related}ⁱ with Pₜᵢₛₛᵤₑⁱ.

In certain embodiments, Pₜᵢₛₛᵤₑⁱ is calculated by evaluating the expression

$${P}_{tissue}^{t} = \frac{\frac{1}{{d}_{k}^{2}}}{{\sum }_{q = 1}^{U} \frac{1}{{d}_{q}^{2}}} \qquad ( 3 )$$

for one or more of the diseases or conditions represented in the plurality of nearest neighbors calculated as described herein, wherein in Eqn. (3) dₖ is the Euclidian distance between the sample data set obtained from a sample from the individual in need of diagnosis and the center of a distinct labeling set representing a disease or condition, and d_{q} is the Euclidean distance between the sample data set and any of the U centers of the distinct labeling set representing the disease or condition. For example without limitation, if a specific disease or condition is associated with a specific tissue, and if a particular secondary SOM displays one of the nearest neighbors found in the procedure described above (i.e., one of the nearest neighbors is found in the tissue type of the specific disease or condition), then d_{q} is the Euclidean distance between the sample data set and the center of each cluster found within the particular secondary SOM.

In certain embodiments, when the plurality of nearest neighbors do not each represent the same disease or condition, P_{related}ⁱ is calculated by evaluating probabilities P_{cluster}ⁱ and Pₜᵢₛₛᵤₑⁱ as described above, and further calculating the probability

P_{related}ⁱ=αP_{cluster}+βPₜᵢₛₛᵤₑ (4)

wherein α+β=1. The proportionality factors α and β can be optimized, for example without limitation, by evaluating the prediction of histologically certified test samples. In certain embodiments, the histologically certified test samples do not form any of the samples used for training the primary SOM. In certain embodiments, α=0.3 and β=0.7.

In certain embodiments, the method for constructing a SOM useful in the diagnosis of an individual suffering from a disease or condition employs the method described herein for construction of a primary SOM, and the formation of at least one secondary SOM employs methods described herein.

In certain embodiments, in the method for constructing a SOM useful in the diagnosis of an individual suffering from a disease or condition, the sample data and data sets of measurements representing a plurality of different diseases or conditions are data vectors of scalars, wherein the scalars are continuous or discrete. In some embodiments, the dimensionality of these data vectors is greater than 2. In some embodiments, the dimensionality of these data vectors is greater than 20. In some embodiments, the dimensionality of these data vectors is at least 29. In some embodiments, the dimensionality of these data vectors is 29. In some embodiments, a plurality of secondary SOMs, each using a different distinct labeling set, are formed.

### EXAMPLES

### Diagnostic for Cancer of Unknown Primary

The expression levels of 87 target genes (Table 2) and 5 housekeeping genes (Table 3) were collected for 221 histologically certified tumor tissue samples, including 36 breast cancer, 32 colorectal cancer, 11 kinase cancer, 14 melanoma cancer, 30 non-small cell lung cancer, 33 ovary cancer, 24 pancreas cancer, 20 prostate cancer, 12 stomach cancer, and 9 small cell lung cancer tissue samples. Gene expression levels were determined by PCR as described herein, which employed the forward and reverse primers and probes tabulated in Table 4.

The expression levels of 87 target genes from all samples were each normalized by subtracting from each of these values the average expression levels of the 5 housekeeping genes for each sample, and further subtracting the average gene expression level for each gene representing all samples. The “average gene expression level” is the average expression level across all 221 samples for one gene. After normalization, a step-wise logistic regression was conducted to find the minimum set of genes that contribute a model to predict each tumor tissue type. The minimum set of genes for the 10 tumor tissue types were then combined, which resulted in 29 unique genes to be used in the diagnostic procedure, listed as follows by GenBank® locus: AA782845, AB038160, AF133587, AF301598, A1309080, A1804745, AI985118, AK027147, AK054605, AW291189, AW473119, AY033998, BC001293, BC001639, BC002551, BC004331, BC006537, BC009084, BC010626, BC012926, BC013117, BC015754, M95585, NM_{—}004062, NM_{—}004063, NM_{—}019894, NM_{—}033229, R45389, and X69699.

TABLE 2
Target genes for CUP diagnosis.

| Locus | Description |
|---|---|
| AA456140 | zx65f08.s1 Soares_total_fetus_Nb2HF8_9w (Homo |
| | sapien) |
| AA745593 | NCI_CGAP_GCB1 (Homo sapien) |
| AA765597 | NCI_CGAP_GCB1 (Homo sapien) |
| AA782845 | Soares_parathyroid_tumor_NbHPA (Homo sapien) |
| AA865917 | NCI_CGAP_GC4 (Homo sapien) |
| AA946776 | NCI_CGAP_Kid5 (Homo sapien) |
| AA993639 | Soares_total_fetus_Nb2HF8_9w (Homo sapien) |
| AB038160 | TMPRSS3d mRNA for serine protease (Homo sapien) |
| AF104032 | L-type amino acid transporter subunit LAT1 (Homo sapien) |
| AF133587 | rhabdoid tumor deletion region protein 1 (Homo sapien) |
| AF301598 | empty spiracles-like protein (EMX2) (Homo sapien) |
| AF332224 | testis protein (Homo sapien) |
| AI041545 | Soares_testis_NHT (Homo sapien) |
| AI147926 | Soares_pregnant_uterus_NbHPU (Homo sapien) |
| AI309080 | NCI_CGAP_Br15 (Homo sapien) |
| AI341378 | NCI_CGAP_GC6 (Homo sapien) |
| AI457360 | NCI_CGAP_Co14 (Homo sapien) |
| AI620495 | NCI_CGAP_Pr28 (Homo sapien) |
| AI632869 | NCI_CGAP_Ut1 (Homo sapien) |
| AI683181 | NCI_CGAP_Ut1 (Homo sapien) |
| AI685931 | NCI_CGAP_Pr28 (Homo sapien) |
| AI802118 | NCI_CGAP_Lu24 (Homo sapien) |
| AI804745 | NCI_CGAP_Pr28 (Homo sapien) |
| AI952953 | NCI_CGAP_GC6 (Homo sapien) |
| AI985118 | NCI_CGAP_Kid11 (Homo sapien) |
| AJ000388 | HSCANPX calpain-like protease(Homo sapien) |
| AK025181 | FLJ21528 fis, clone COL05977 (Homo sapien) |
| AK027147 | FLJ23494 fis, clone LNG01885 (Homo sapien) |
| AK054605 | FLJ30043 fis, clone 3NB692001548 (Homo sapien) |
| AL023657 | HSDSHP (Homo sapien) SH2D1A cDNA, (Homo sapien) |
| AL039118 | DKFZp566J244_s1 566 (synonym: hfkd2) (Homo sapien) |
| AL110274 | DKFZp564I0272 (Homo sapien) |
| AL157475 | DKFZp761G151 (Homo sapien) |
| AW118445 | NCI_CGAP_Brn35 (Homo sapien) |
| AW194680 | NCI_CGAP_Kid13 (Homo sapien) |
| AW291189 | NCI_CGAP_Sub4 (Homo sapien) |
| AW298545 | NCI_CGAP_Sub6 (Homo sapien) |
| AW445220 | NCI_CGAP_Sub5 (Homo sapien) |
| AW473119 | NCI_CGAP_Ut1 (Homo sapien) |
| AY033998 | HUDPRO1 (Homo sapien) |
| BC000045 | vestigial like 1 Drosophila (Homo sapien) |
| BC001293 | homeobox C10 (Homo sapien) |
| BC001504 | pyrroline-5-carboxylate reductase 1 (Homo sapien) |
| BC001639 | solute carrier family 43, member 1 (Homo sapien) |
| BC002551 | cell division cycle associated 3 (Homo sapien) |
| BC004331 | hydroxysteroid dehydrogenase like 2 (Homo sapien) |
| BC004453 | 5-hydroxytryptamine (serotonin) receptor 3A (Homo |
| | sapien) |
| BC005364 | chromosome 10 open reading frame 59 (Homo sapien) |
| BC006537 | homeobox A9 (Homo sapien) |
| BC006811 | peroxisome proliferative activated receptor (Homo sapien) |
| BC006819 | S100 calcium binding protein P (Homo sapien) |
| BC008764 | kinesin family member 2C (Homo sapien) |
| BC008765 | syndecan 1 (Homo sapien) |
| BC009084 | selenium binding protein 1 (Homo sapien) |
| BC009237 | thyroid stimulating hormone receptor (Homo sapien) |
| BC010626 | kinesin family member 12 (Homo sapien) |
| BC011949 | carbonic anhydrase II (Homo sapien) |
| BC012926 | EPS8-like 3 (Homo sapien) |
| BC013117 | regulator of G-protein signalling 17 (Homo sapien) |
| BC015754 | Ca2+dependent secretion activator (Homo sapien) |
| BC017586 | calcyphosine-like (Homo sapien) |
| BE552004 | NCI_CGAP_GC6 (Homo sapien) |
| BE962007 | NIH_MGC_65 (Homo sapien) |
| BF224381 | NCI_CGAP_Lu24 (Homo sapien) |
| BF437393 | NCI_CGAP_Pr28 (Homo sapien) |
| BF446419 | NCI_CGAP_Lu24 (Homo sapien) |
| BF592799 | NCI_CGAP_GC6 (Homo sapien) |
| BI493248 | Morton Fetal Cochlea (Homo sapien) |
| H05388 | Soares infant brain 1NIB (Homo sapien) |
| H07885 | Soares infant brain 1NIB (Homo sapien) |
| H09748 | Soares infant brain 1NIB (Homo sapien) |
| M95585.1 | Human hepatic leukemia factor (Homo sapien) |
| N64339 | Morton Fetal Cochlea (Homo sapien) |
| NM_000065 | complement component 6 (Homo sapien) |
| NM_001337 | chemokine (C—X3—C motif) receptor 1 (Homo sapien) |
| NM_003914 | cyclin A1 (Homo sapien) |
| NM_004062 | cadherin 16 (Homo sapien) |
| NM_004063 | cadherin 17 (Homo sapien) |
| NM_004496 | forkhead box A1 (Homo sapien) |
| NM_006115 | preferentially expressed antigen in melanoma (PRAME), |
| | transcript variant 1 (Homo sapien) |
| NM_019894 | transmembrane protease, serine 4 (TMPRSS4), transcript |
| | variant (Homo sapien) |
| NM_033229 | tripartite motif-containing 15 (TRIM15), transcript variant |
| | 1(Homo sapien) |
| R15881 | Soares infant brain 1NIB (Homo sapien) |
| R45389 | Soares infant brain 1NIB (Homo sapien) |
| R61469 | Soares infant brain 1NIB (Home sapien) |
| X69699 | Pax8 (Homo sapien) |
| X96757 | MAP kinase kinase (Home sapien) |

TABLE 3
Housekeeping genes for CUP diagnosis

| Locus | Description |
|---|---|
| BC006091 | TSSC4, tumor suppressing subtransferable candidate 4 |
| AL137727 | TMEM55B, transmembrane protein 55B |
| BC016680 | SP2, Sp2 transcription factor |
| BC003043 | ARF5, ADP-ribosylation factor 5 |
| AF308803 | VPS33B, vacuolar protein sorting 33B |

TABLE 4
Genes, forward primers, reverse primers, and probes for CUP diagnosis.

| | Forward Primer | Reverse Primer | Probe |
|---|---|---|---|
| Locus | (5′-3′) | (5′-3′) | (5′-3′) |
| AA456140 | CAGTCTAGACATGCTGCAAGGAA | TGTGCGTTCAAGAAAGGATATG | AACGGACTTTAGAATCTTCT |
| | (SEQ ID NO: ) | GAA | (SEQ ID NO: ) |
| | | (SEQ ID NO: ) | |
| AA745593 | CCTGGAGACCCGGAGACA | AGTCGTGACAGTTCCCGTGTT | AGGCCTGGACAAGGA |
| | (SEQ ID NO: ) | (SEQ ID NO: ) | (SEQ ID NO: ) |
| AA765597 | TTGTACTGAGCTGTGAAGTCAGTGTT | GCCACCATCCAAACCTCAAT | AGTTTATTCATGGAGCATGC |
| | (SEQ ID NO: ) | (SEQ ID NO: ) | (SEQ ID NO: ) |
| AA782845 | CCGCGGTGTACAATACCCATA | GGAAGTAAAAGCAGCCAGCAAT | ACATTGTGCAGGAGGG |
| | (SEQ ID NO: ) | (SEQ ID NO: ) | (SEQ ID NO: ) |
| AA865917 | CCCTTACATTCTGCACTTCATAGTTG | CCCTTTCCAAGTCCCTCCAT | CTGAGCTTAGGATCATC |
| | (SEQ ID NO: ) | (SEQ ID NO: ) | (SEQ ID NO: ) |
| AA946776 | GGCGGAGCGAGAGCAAA | CTGATCAGAAATGAAAAGCGTG | CATCAGGCCGCAGTCC |
| | (SEQ ID NO: ) | TCTT | (SEQ ID NO: ) |
| | | (SEQ ID NO: ) | |
| AA993639 | TGTGCCTCCTCTTAGCATCTGTT | GGCAGGCATTTTATTCATCATTT | CTGACTCCCAGTTATTT |
| | (SEQ ID NO: ) | (SEQ ID NO: ) | (SEQ ID NO: ) |
| A8038160 | GAGAAGATTGTCTACCACAGCAAGT | CAGCTTCATAAGGGCGATGTCA | TTGCCCAGCCTCTTTG |
| | (SEQ ID NO: ) | (SEQ ID NO: ) | (SEQ ID NO: ) |
| AF104032 | CCAGCGGTTTCCACTTGTG | CACAACGACTGAAAATGCACTTG | TTTTCAAGCACAACCC |
| | (SEQ ID NO: ) | (SEQ ID NO: ) | (SEQ ID NO: ) |
| AF133587 | TCAAGTGGCCGAAGCCTTAC | GGCTCAGGGTTTGAACTCGAT | CCGGATCGCCATCAG |
| | (SEQ ID NO: ) | (SEQ ID NO: ) | (SEQ ID NO: ) |
| AF301598 | GGCAAGTTTTCAAGCACTGAGTT | ACATTAAGGAAGCATTTGTCAC | TTCCAGATCATAGACTTAC |
| | (SEQ ID NO: ) | TCTCT | (SEQ ID NO: ) |
| | | (SEQ ID NO: ) | |
| AF332224 | CATTCTCAACAGGGAAACCCTACT | TCCCATGATTCTTCAAAAAGTT | ACTTTGTAAAGCAAATAATG |
| | (SEQ ID NO: ) | CTGTATCTT | (SEQ ID NO: ) |
| | | (SEQ ID NO: ) | |
| AI041545 | AGACCATCGCCAGCATCTG | TGCCTTTGCTGTGGTAAGAATTC | CCTTCAGGGTGTTCGG |
| | (SEQ ID NO: ) | (SEQ ID NO: ) | (SEQ ID NO: ) |
| AI147926 | TGAACAAGATGAACCAATGTGGAT | CCTTTAACAATGTCTGGATATT | AAAGAAGTCCGAGATATT |
| | (SEQ ID NO: ) | TTGGA | (SEQ ID NO: ) |
| | | (SEQ ID NO: ) | |
| AI309080 | GACCCTTGGAGCAGTGTTGTG | GAGGCTTTATTGACAACGGAGAAG | AACTTGCCTAGAACTC |
| | (SEQ ID NO: ) | (SEQ ID NO: ) | (SEQ ID NO: ) |
| AI341378 | GCCAAAACACTACAAGCCTCTTG | ATCACAAAAATTAGTAAGCCTG | TTTCACCAAAACCC |
| | (SEQ ID NO: ) | AGATGT | (SEQ ID NO: ) |
| | | (SEQ ID NO: ) | |
| AI457360 | AGACACTGTCACCCCCTTTCC | CAGCGAACATCTCTGCTTCATC | CCACAAGACTGGCAGAG |
| | (SEQ ID NO: ) | (SEQ ID NO: ) | (SEQ ID NO: ) |
| AI620495 | GCACACTGAGTCTTAGCGTTTCTG | CAACTGGGCTTGGCGTTATT | TGGAAACAGTTTGGATTGTA |
| | (SEQ ID NO: ) | (SEQ ID NO: ) | (SEQ ID NO: ) |
| AI632869 | CTGGAACCAGCTCTCTCCTAATATTC | TGACTTGGCAATGTAAGACACACA | TTGTGCCCCACACTAAC |
| | (SEQ ID NO: ) | (SEQ ID NO: ) | (SEQ ID NO: ) |
| AI683181 | CCTGTCAAGATTGCAAGAACATGT | GCTGCTTCGGAACAATATAACGT | AAATGTACGGAGCTTCAT |
| | (SEQ ID NO: ) | (SEQ ID NO: ) | (SEQ ID NO: ) |
| AI685931 | CAAATCCTCCTGCCTGAAGAAG | CTGGTTCTCCCCACAAATGC | TCAGCATCACTTCAGC |
| | (SEQ ID NO: ) | (SEQ ID NO: ) | (SEQ ID NO: ) |
| AI802118 | CCGCTCCTGCAAATTGAGAT | CACACATTGTCTCTAATCCTTA | ATGCCTGCCTTTCAA |
| | (SEQ ID NO: ) | CAATGAC | (SEQ ID NO: ) |
| | | (SEQ ID NO: ) | |
| AI804745 | GGCACCCCGCATTCG | TCCACCCCCCAAAATCAAC | TGTGAGGTTTGTTTGTCC |
| | (SEQ ID NO: ) | (SEQ ID NO: ) | (SEQ ID NO: ) |
| AI952953 | TCACGATGATCCTGACAATGC | CAAAGTGCCCTTCTGCTCCTT | CATGAGAGCCCAGAACA |
| | (SEQ ID NO: ) | (SEQ ID NO: ) | (SEQ ID NO: ) |
| AI985118 | TTTCTAGTGAGCTAACCGTAACA | CACAACGATCTTCTACACGTGACA | CCTACAGGATACACGTGAGA |
| | GAGA | (SEQ ID NO: ) | (SEQ ID NO: ) |
| | (SEQ ID NO: ) | | |
| AJ000388 | GCCTACCTAGACCAGCAAGCAT | AGTTAAACAGACTGGAAAACAT | CATTTTTAGCTCGCTCATT |
| | (SEQ ID NO: ) | GGTAAA | (SEQ ID NO: ) |
| | | (SEQ ID NO: ) | |
| AK025181 | GCACCGCTGGATGAAAGG | CCTTTGTTTGTTAACTGCTCTTTCC | AGGCTAGAGGCTGAGGG |
| | (SEQ ID NO: ) | (SEQ ID NO: ) | (SEQ ID NO: ) |
| AK027147 | GAGAGGAAGAATTGCAGAGTAGT | CCAAAGAACAGACATGCAGTTATTG | ATCATGCCAATTCC |
| | TTGT | (SEQ ID NO: ) | (SEQ ID NO: ) |
| | (SEQ ID NO: ) | | |
| AK054605 | CAAGGATTTTTCCAGGCACAGT | ACCTTGGCCTCTCCAAGCA | CATACCTGTAATCCC |
| | (SEQ ID NO: ) | (SEQ ID NO: ) | (SEQ ID NO: ) |
| AL023657 | CCATGTACTGGCAAGACCTGATT | CAGGCCACACTCCACTTTTGT | TATGGATGCCGTGGGAG |
| | (SEQ ID NO: ) | (SEQ ID NO: ) | (SEQ ID NO: ) |
| AL039118 | GCGCAAATGCCGCATAA | GCATATGACCACAGTATCACAA | TTGAGTGATTGTTAATGTTGTCT |
| | (SEQ ID NO: ) | TCAA | (SEQ ID NO: ) |
| | | (SEQ ID NO: ) | |
| AL110274 | CCTCCTGTAGCATGTGTCCAAGT | TCACATTTTTTGTTGCAGTCCAA | AGCCACTAACCAACTAG |
| | (SEQ ID NO: ) | (SEQ ID NO: ) | (SEQ ID NO: ) |
| AL157475 | GTGCTGTTTGCAGTTGTACTCATT | GTTTTACACCCAGCGATGCTT | CTCTCTGCCATCCCC |
| | (SEQ ID NO: ) | (SEQ ID NO: ) | (SEQ ID NO: ) |
| AW118445 | TTCCAGACTTGTCACTGACTTTCCT | CTGCCCACAGCCTCTTTTTC | CTGGAGCAGGTGGC |
| | (SEQ ID NO: ) | (SEQ ID NO: ) | (SEQ ID NO: ) |
| AW194680 | AAGGCGCTGGTGTTTTGCT | AATAACCTGCATTCACCGAAGAG | TGAGTTTTAAGAGATCCC |
| | (SEQ ID NO: ) | (SEQ ID NO: ) | (SEQ ID NO: ) |
| AW291189 | GCCCGGATGAAGCATGAGAT | CCGCTACACGTTGGTGCTA | TTCACGCACTGTCCCTC |
| | (SEQ ID NO: ) | (SEQ ID NO: ) | (SEQ ID NO: ) |
| AW298545 | CCCTTCCCTCAATTTCCTGTTT | AGGAATCTCCGAGTTGAGGAAAA | AAACTGAATGGCACGAAA |
| | (SEQ ID NO: ) | (SEQ ID NO: ) | (SEQ ID NO: ) |
| AW445220 | CACGGGACTGCCACAGA | ACAAGTTTAATGCAACAGGTGA | ATGCTCCGGAAGGCTCA |
| | (SEQ ID NO: ) | CAAC | (SEQ ID NO: ) |
| | | (SEQ ID NO: ) | |
| AW473119 | CAATGCTTTTTGTGCACTACATA | ACAATTTGGCATTTGAGCCTTTTCC | CAGTGTAGAGCTCTTGTTTTA |
| | CTCT | (SEQ ID NO: ) | (SEQ ID NO: ) |
| | (SEQ ID NO: ) | | |
| AY033998 | CACACATACACGAAAGAGAGAGA | AACACTGGCTTATAAAGTCCATGGT | ACTTTTCAAGGCTTATATTC |
| | AACA | (SEQ ID NO: ) | (SEQ ID NO: ) |
| | (SEQ ID NO: ) | | |
| BC000045 | AAGACACGGCAGCAAGACATC | CAAGTGGGTGTGAGCAGCTTT | CTGCATATTGTTCCAGATAA |
| | (SEQ ID NO: ) | (SEQ ID NO: ) | (SEQ ID NO: ) |
| BC001293 | CATAGCAAAGCAAAGACAGAATGC | AATATCTTTAAATAACACAACT | CCCCCCAAATATT |
| | (SEQ ID NO: ) | CCCAGACA | (SEQ ID NO: ) |
| | | (SEQ ID NO: ) | |
| BC001504 | GTGGAATAGTGGAGGCCTTCAA | GCAGATGCCCTCCAAGATGT | TGATTAGACAAGGCCC |
| | (SEQ ID NO: ) | (SEQ ID NO: ) | (SEQ ID NO: ) |
| BC001639 | GCATGTGTCTGTGTATGTGTGAATGT | AGGCCCCTTTCCTTCTGAAA | AGAGACACAGCCCTC |
| | (SEQ ID NO: ) | (SEQ ID NO: ) | (SEQ ID NO: ) |
| BC002551 | CCAGGACCATGACAAGGAAAAT | GCCATGCAGGGCCTAGCT | AGCACTTTCCCTTGTG |
| | (SEQ ID NO: ) | (SEQ ID NO: ) | (SEQ ID NO: ) |
| BC004331 | TGGCGGGGCTTCTGTTTTATTT | TGGCTTTTATTAGCGATTCATGAA | TAGGCTGGATGCTACCCA |
| | (SEQ ID NO: ) | (SEQ ID NO: ) | (SEQ ID NO: ) |
| BC004453 | GATAACTCTGTACGAGGCTTCTC | AGGGAAGCTGCCACAAGTGA | CTAGTGTCTTTTTTTTCTTCAC |
| | TAACC | (SEQ ID NO: ) | (SEQ ID NO: ) |
| | (SEQ ID NO: ) | | |
| BC005364 | AATTCCTCACACCTTGCACCTT | TTTTAAGTACCACTTTTCCTCC | ACTTTTCTGAATTGCTATGACT |
| | (SEQ ID NO: ) | AACAA | (SEQ ID NO: ) |
| | | (SEQ ID NO: ) | |
| BC006537 | AAACCGCCATTGGGCTACT | AGTGTAAGTTCAGTCTGATGGA | CATCAAGGATACAAATCTAC |
| | (SEQ ID NO: ) | AACC | (SEQ ID NO: ) |
| | | (SEQ ID NO: ) | |
| BC006811 | AGAAGACGGAGACAGACATGAGT | CTCAGGACTCTCTGCTAGTACAAGT | CCCGCTCCTGCAGGAG |
| | (SEQ ID NO: ) | (SEQ ID NO: ) | (SEQ ID NO: ) |
| BC006819 | TGCAGAGTGGAAAAGACAAGGAT | TGGCGTCCAGGTCCTTGA | CCGTGGATAAATTG |
| | (SEQ ID NO: ) | (SEQ ID NO: ) | (SEQ ID NO: ) |
| BC008764 | GGGAGAGAGACGGAGCCTTTA | GCCCAAAGGCGTAGAAGGTT | ACAGCTATCTGCTGGCT |
| | (SEQ ID NO: ) | (SEQ ID NO: ) | (SEQ ID NO: ) |
| BC008765 | CTGGGCTGGAATCAGGAATATTT | GGATTAAGTAGAGTTTTGCCAA | CCAAAGAGTGATAGTCTTT |
| | (SEQ ID NO: ) | AAGC | (SEQ ID NO: ) |
| | | (SEQ ID NO: ) | |
| BC009084 | CGATTGTAGCTCTGACATCTGGATT | GGGCCCAAAATAGGGAGTGT | TCCACCCTCATCACCC |
| | (SEQ ID NO: ) | (SEQ ID NO: ) | (SEQ ID NO: ) |
| BC009237 | TGCCTGGCACAAAGAAGGA | CCCCATGATTGTAAGTTCTTCCA | AAATGATAGTTCGACTCGTCT |
| | (SEQ ID NO: ) | (SEQ ID NO: ) | (SEQ ID NO: ) |
| BC010626 | ACCCAGGAGACTGCTGTGTGA | CATTCAGCAGATGGGCAGACT | CTCCACACTCTTGGGC |
| | (SEQ ID NO: ) | (SEQ ID NO: ) | (SEQ ID NO: ) |
| BC011949 | AAATGCTGCTTTTAAAACATAGGAAA | TGCCTTAACTAGCTCAATTTAT | TAGAATGGTTGAGTGCAAAT |
| | (SEQ ID NO: ) | CTTGTG | (SEQ ID NO: ) |
| | | (SEQ ID NO: ) | |
| BC012926 | GGCCCCGCTGATGCA | TGCTGCAAACTGGGATCCA | ATGGCAGATCTGATACCC |
| | (SEQ ID NO: ) | (SEQ ID NO: ) | (SEQ ID NO: ) |
| BC013117 | GAGCTATTTATCTCTGTTTGTTG | CCACAGTTTTGGCAGTGAACAA | CCAGAGGAATCCCC |
| | GAAAATCC | (SEQ ID NO: ) | (SEQ ID NO: ) |
| | (SEQ ID NO: ) | | |
| BC015754 | CATTTTGATCTGTAACTGCACAACCC | CAAGATGGATCCACTACTTTAC | CTGCAGCAAACCCCA |
| | (SEQ ID NO: ) | ATGGA | (SEQ ID NO: ) |
| | | (SEQ ID NO: ) | |
| BC017586 | CCATGTGGCTCCAAATGACTAA | TTAGGATGAGTGTGAAATCAAA | TGTCAGCTCAAAAACCAGA |
| | (SEQ ID NO: ) | TACGA | (SEQ ID NO: ) |
| | | (SEQ ID NO: ) | |
| BE552004 | AGGCCCAGGTTTCGACAGA | GGCTCCGAAATGGCATCTC | AGGGAGAGAAAACC |
| | (SEQ ID NO: ) | (SEQ ID NO: ) | (SEQ ID NO: ) |
| BE962007 | GTGAGAAACTGAATGTATTATTC | GTGCAAATTGACTTTTACATTC | ACTGAGTGCCTTCATTT |
| | AGGAAGA | AACTTTAG | (SEQ ID NO: ) |
| | (SEQ ID NO: ) | (SEQ ID NO: ) | |
| BF224381 | ACGCCACAGGAGGACATGTT | TCACACCCCCATACTCTTCTGTT | CTGCAGATGTAGTTGCC |
| | (SEQ ID NO: ) | (SEQ ID NO: ) | (SEQ ID NO: ) |
| BF437393 | CGCTGTGGGCAATTGTTACA | CCCATAAAGCAATTCACGGATACAG | TTCACAGTAAACCTAAGAACACT |
| | (SEQ ID NO: ) | (SEQ ID NO: ) | (SEQ ID NO: ) |
| BF446419 | AGCTCCACAACCCTGTTTGG | GCTTGGGAAACCGCACTTT | ACTGCAGGACCAGAAG |
| | (SEQ ID NO: ) | (SEQ ID NO: ) | (SEQ ID NO: ) |
| BF592799 | GCCATGACTGGTGATTTCATGA | ATGCATGGGCCATTGATCTT | CCTCCGTAGGCATCA |
| | (SEQ ID NO: ) | (SEQ ID NO: ) | (SEQ ID NO: ) |
| BI493248 | AAATGTGTAGTTTCTTAATCGCA | GGTCACATAAAAATACATGAGG | TGCAACACTGTGTATTAG |
| | CTACCT | ATGATAA | (SEQ ID NO: ) |
| | (SEQ ID NO: ) | (SEQ ID NO: ) | |
| H05388 | ACAGGTTCTTATCTGCAAGGTTCAA | TGACTGGCCCTGCAGAATACT | TTGCTTAGACATTGTTTTC |
| | (SEQ ID NO: ) | (SEQ ID NO: ) | (SEQ ID NO: ) |
| H07885 | GTCACTGTCATAGCAGCTGTGATTT | CCCACTCCCCATCAACCA | CAAGGAAGGGTGCTGCA |
| | (SEQ ID NO: ) | (SEQ ID NO: ) | (SEQ ID NO: ) |
| H09748 | TGTACAAGATTTTGGGCCTCTTTT | AAATGGACAGACACATGCTGAACT | TCCTTAATGTCACAATGTT |
| | (SEQ ID NO: ) | (SEQ ID NO: ) | (SEQ ID NO: ) |
| M95585 | TTGTAACATGGACCATCCAAATTTAT | CCAAGAGAGACCAGTGCTCAAATA | CAAATGGTAGCTGAAAAA |
| | (SEQ ID NO: ) | (SEQ ID NO: ) | (SEQ ID NO: ) |
| N64339 | GCTTTCTGAATGTAGACGGAACAGT | TTGGCAAACGGATGAGTTAAAAA | TGGAAGCAGAAGGC |
| | (SEQ ID NO: ) | (SEQ ID NO: ) | (SEQ ID NO: ) |
| NM_000065 | TCTTCAATGAGTTAATAAACAGA | TGAATGAAGATATGAAAGCTGG | CCTCTGAAACACATTCTTG |
| | AATCTCCAGAA | GCTT | (SEQ ID NO: ) |
| | (SEQ ID NO: ) | (SEQ ID NO: ) | |
| NM_001337 | GTTAGACCACAAATAGTGCTCGCT | ATGAATACACAGTCTGGTAGAG | TTCTATGTAGTTTGGTAATTATCA |
| | (SEQ ID NO: ) | TCTTCT | (SEQ ID NO: ) |
| | | (SEQ ID NO: ) | |
| NM_003914 | TTCCAGAACTTCACCTCCATATCA | GATCCAACGTGCAGAAGCCTAT | AGTGCCAATAATCG |
| | (SEQ ID NO: ) | (SEQ ID NO: ) | (SEQ ID NO: ) |
| NM_004062 | GCCTGGACACCAACTTTATGG | GGGCTTTATTATTGGGCAAACA | AGTGCTCCAAATGTC |
| | (SEQ ID NO: ) | (SEQ ID NO: ) | (SEQ ID NO: ) |
| NM_004063 | CAAACACAACCTACTCTGCAAACC | GCATGGCAGGTAGTGAGGAAA | AAAGGAACCAGTCAGCTG |
| | (SEQ ID NO: ) | (SEQ ID NO: ) | (SEQ ID NO: ) |
| NM_004496 | CATTGCCATCGTGTGCTTGT | ACCCTCTGGCTATACTAACACC | CAGTGTTATGCACTTTC |
| | (SEQ ID NO: ) | AACT | (SEQ ID NO: ) |
| | | (SEQ ID NO: ) | |
| NM_006115 | GATTCTGGCTTGGGAAGTACATG | GCTTCTCTTTATTTTCAACAGT | AATCCCTGTGTAGACTGT |
| | (SEQ ID NO: ) | TTCTTTAC | (SEQ ID NO: ) |
| | | (SEQ ID NO: ) | |
| NM_019894 | CCCACACTACTGAATGGAAGCA | CCTCTCCAGCCCACAGTGAT | CTGTCTTGTAAAAGCC |
| | (SEQ ID NO: ) | (SEQ ID NO: ) | (SEQ ID NO: ) |
| NM_033229 | GCGTGAGGCGAGAGAACAG | GAGCTGAGGGCCTAAGATAAAT | AGTCTCGAACAGCGGTT |
| | (SEQ ID NO: ) | AAAGT | (SEQ ID NO: ) |
| | | (SEQ ID NO: ) | |
| R15881 | TCAGAACCCACTTTCAAGATGCT | GCTGCTTGCGCCTCTTTTT | TGCTGTGCCAGTGTGA |
| | (SEQ ID NO: ) | (SEQ ID NO: ) | (SEQ ID NO: ) |
| R45389 | AGTGGATCAGACAGTACGACTTTGA | TCCAAAGCAGCTTAGGTGAAAAA | CTGGTGAATGTAAACAAT |
| | (SEQ ID NO: ) | (SEQ ID NO: ) | (SEQ ID NO: ) |
| R61469 | TTCCCCGGGCATTTGTT | CATGTCGCAGGGTTAAGTATGATG | TTCAAACAGACTTTAACCTC |
| | (SEQ ID NO: ) | (SEQ ID NO: ) | (SEQ ID NO: ) |
| X69699 | TGTTTGGGTCAAGCTTCCTTCT | GGCAAAGAGAGACATTTCACTCAGA | CCCCCAGACTTTGG |
| | (SEQ ID NO: ) | (SEQ ID NO: ) | (SEQ ID NO: ) |
| X96757 | CCCTGCCTCTCAGAGGGTTT | ATTCCAAGGCCCCCTTAAGA | CTCTCCCAATTTTC |
| | (SEQ ID NO: ) | (SEQ ID NO: ) | (SEQ ID NO: ) |

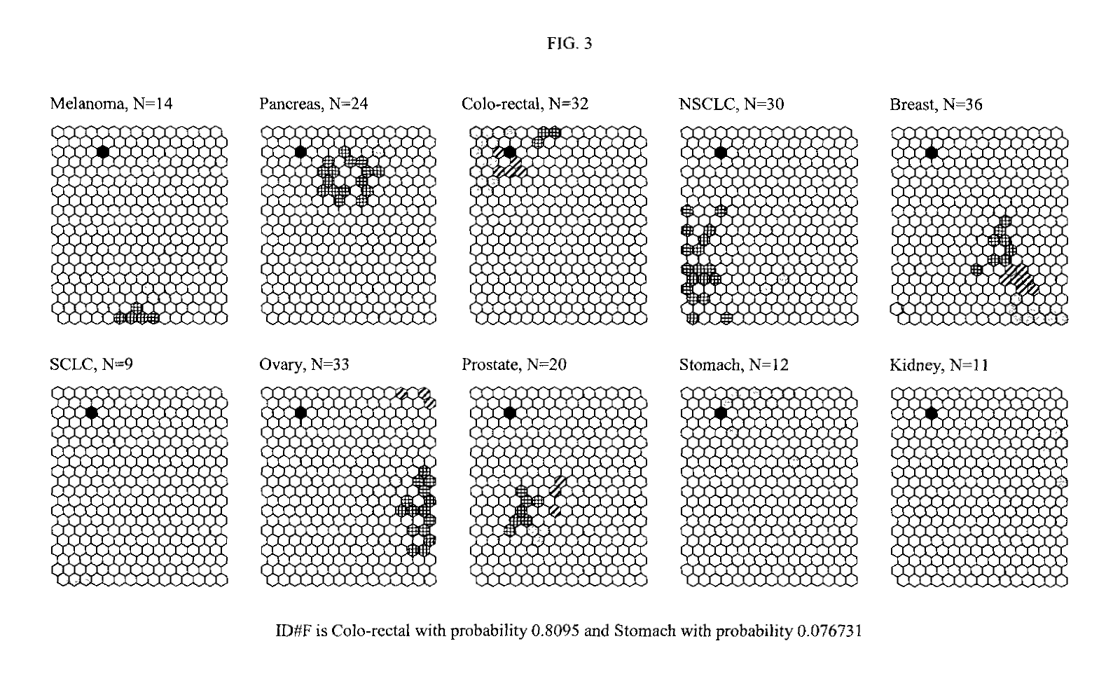

A primary SOM was constructed by the methods described herein using the 29 gene set normalized gene expression data described above. Additionally, a metastatic site of an individual in need of diagnosis was biopsied, and the gene expression data obtained therefrom (i.e., sample data set) was used with the primary SOM to form various secondary SOMs as shown in FIG. 3. In FIG. 3, the map cell in each secondary SOM most similar to the gene expression of the individual needing diagnosis is indicated (i.e., solid black filled hexagon). In this case, the 3 nearest neighbors (i.e., individual tissue samples with lowest Euclidean distance) of the sample data set belong to two different tissue types, colorectal and stomach. Accordingly, the probability of origin of the cancer of the metastatic site was calculated using Eqn. (4). In this example, the sample is predicted to be colorectal cancer with a probability of 81%, and stomach with a probability of 8%, using α=0.3 and β=0.7.

### Therapy Response Profiling

The invention provides methods of therapy response profiling using the methods of SOM construction and display as described herein. As used herein, “therapy response profile” refers to the pattern of expression of a group of genes of a particular tissue type in a particular disease or condition, which pattern is labeled with a distinct labeling set according to the response of the disease or condition to a particular agent or therapeutic regimen. Therapy response profiling can be used to determine if a particular disease or condition will be susceptible to a particular agent or therapeutic regimen.

Thus, gene expression levels of a plurality of samples of tissues having a known disease or condition can be collected and used to construct a primary SOM by the methods described herein. The results of subsequent therapeutic intervention (e.g., administration of a particular drug) in each case can then be used to construct a distinct labeling set which characterizes the efficacy of such therapeutic interventions. For example, if a particular disease or condition does not respond to a particular agent or therapeutic regimen, the distinct label for the disease or condition to the agent or therapeutic regimen would be for example “non-responsive.” Alternatively, if a particular disease or condition responds very well to a particular agent or therapeutic regimen, the distinct label for the disease or condition would be labeled “highly responsive.” Intermediate states of response (e.g., “low response,” “intermediate response” and the like) may be employed in the construction of the distinct labeling sets.

When a sample from a subject suffering from the disease or condition used to train the primary SOM is analyzed for gene expression levels, the gene expression pattern so obtained can be used to form a plurality of secondary SOMs, each having a different distinct labeling set, wherein each distinct labeling set characterizes a particular therapeutic regimen. Then, by inspection of the distinct labeling set of each secondary SOM, a prediction can be drawn on the susceptibility of the underlying disease or condition to a particular therapeutic regimen. For example, if the unknown sample mapped near a known sample having a favorable response to a particular drug, then that drug would be indicated for therapeutic intervention for the underlying disease or condition. In one embodiment, the therapy response profile may be applied to cancer as the disease or condition.

### Therapy Response Information

The invention provides methods of providing therapy response information using the methods of SOM construction and display as described herein. As used herein, “therapy response information” refers to annotation describing the historic result of therapeutic intervention in a disease or condition of one or more samples used to provide the plurality of data sets of measurements used to construct a primary SOM. Examples of therapy response information include previous therapeutic regimens (e.g., drugs administered and the like) and responses thereto. In some embodiments, after a map cell in a primary or second SOM is picked, therapy response information associated with the picked map cell, and optionally associated with nearby map cells, is displayed. Thus, by picking the map cell in a primary or secondary SOM representing the individual in need of diagnosis, the clinician is provided with information on the efficacy of various drugs and other therapeutic regimens with respect to the underlying disease or condition.

### Autoimmune Disorder Diagnosis

The invention provides methods for diagnosis of autoimmune disorders using the methods of SOM construction and display as described herein. Autoimmune disorders occur when the normal control processes for differentiating self from non-self are disrupted. Such disorders result in a variety of conditions, including destruction of one or more types of body tissues, abnormal growth of an organ, or changes in organ function. Examples of autoimmune disorders include without limitation Hashimoto's thyroiditis, pernicious anemia, Addison's disease, type I diabetes, rheumatoid arthritis, systemic lupus erythematosus, dermatomyositis, Sjorgren's syndrome, lupus erythematosus, multiple sclerosis, myasthenia gravis, Reiter's syndrome, Grave's disease, and celiac disease.

In one embodiment, the expression levels of genes associated with a plurality of autoimmune disorders could be obtained by methods described herein, which gene expression levels could then be used to construct a primary SOM. Such genes may include, for example, genes encoding MHC (i.e., major histocompatibility complex) antigen (Shirai, Tohoku J. Exp. Med., 1994, 173:133-40). In this case, the distinct labeling sets as described herein corresponds to each specific autoimmune disease. One or more secondary SOMs could be formed using the gene expression levels of an individual suspected of suffering from an autoimmune disorder. Visualization of one or more of the secondary SOMs then provides assistance in the diagnosis of a specific autoimmune disease by methods described herein.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses which will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any two different values as the endpoints of a range. Such ranges are also within the scope of the described invention.

Thus, additional embodiments are within the scope of the invention and within the following claims.

TABLE 4
Genes, forward primers, reverse primers, and probes for CUP diagnosis.

| | Forward Primer | Reverse Primer | Probe |
|---|---|---|---|
| Locus | (5′-3′) | (5′-3′) | (5′-3′) |
| AA456140 | CAGTCTAGACATGCTGCAAGGAA | TGTGCGTTCAAGAAAGGATATG | AACGGACTTTAGAATCTTCT |
| | (SEQ ID NO: 1) | GAA | (SEQ ID NO: 175) |
| | | (SEQ ID NO: 88) | |
| AA745593 | CCTGGAGACCCGGAGACA | AGTCGTGACAGTTCCCGTGTT | AGGCCTGGACAAGGA |
| | (SEQ ID NO: 2) | (SEQ ID NO: 89) | (SEQ ID NO: 176) |
| AA765597 | TTGTACTGAGCTGTGAAGTCAGT | GCCACCATCCAAACCTCAAT | AGTTTATTCATGGAGCATGC |
| | GTT | (SEQ ID NO: 90) | (SEQ ID NO: 177) |
| | (SEQ ID NO: 3) | | |
| AA782845 | CCGCGGTGTACAATACCCATA | GGAAGTAAAAGCAGCCAGCAAT | ACATTGTGCAGGAGGG |
| | (SEQ ID NO: 4) | (SEQ ID NO: 91) | (SEQ ID NO: 178) |
| AA865917 | CCCTTACATTCTGCACTTCATAG | CCCTTTCCAAGTCCCTCCAT | CTGAGCTTAGGATCATC |
| | TTG | (SEQ ID NO: 92) | (SEQ ID NO: 179) |
| | (SEQ ID NO: 5) | | |
| AA946776 | GGCGGAGCGAGAGCAAA | CTGATCAGAAATGAAAAGCGTG | CATCAGGCCGCAGTCC |
| | (SEQ ID NO: 6) | TCTT | (SEQ ID NO: 180) |
| | | (SEQ ID NO: 93) | |
| AA993639 | TGTGCCTCCTCTTAGCATCTGTT | GGCAGGCATTTTATTCATCATTT | CTGACTCCCAGTTATTT |
| | (SEQ ID NO: 7) | (SEQ ID NO: 94) | (SEQ ID NO: 181) |
| AB038160 | GAGAAGATTGTCTACCACAGCAA | CAGCTTCATAAGGGCGATGTCA | TTGCCCAGCCTCTTTG |
| | GT | (SEQ ID NO: 95) | (SEQ ID NO: 182) |
| | (SEQ ID NO: 8) | | |
| AF104032 | CCAGCGGTTTCCACTTGTG | CACAACGACTGAAAATGCACTTG | TTTTCAAGCACAACCC |
| | (SEQ ID NO: 9) | (SEQ ID NO: 96) | (SEQ ID NO: 183) |
| AF133587 | TCAAGTGGCCGAAGCCTTAC | GGCTCAGGGTTTGAACTCGAT | CCGGATCGCCATCAG |
| | (SEQ ID NO: 10) | (SEQ ID NO: 97) | (SEQ ID NO: 184) |
| AF301598 | GGCAAGTTTTCAAGCACTGAGTT | ACATTAAGGAAGCATTTGTCAC | TTCCAAGATCATAGACTTAC |
| | (SEQ ID NO: 11) | TCTCT | (SEQ ID NO: 185) |
| | | (SEQ ID NO: 98) | |
| AF332224 | CATTCTCAACAGGGAAACCCTACT | TCCCATGATTCTTCAAAAAGTT | ACTTTGTAAAGCAAATAATG |
| | (SEQ ID NO: 12) | CTGTATCTT | (SEQ ID NO: 186) |
| | | (SEQ ID NO: 99) | |
| AI041545 | AGACCATCGCCAGCATCTG | TGCCTTTGCTGTGGTAAGAATTC | CCTTCAGGGTGTTCGG |
| | (SEQ ID NO: 13) | (SEQ ID NO: 100) (SEQ ID | NO: 187) |
| AI147926 | TGAACAAGATGAACCAATGTGGAT | CCTTTAACAATGTCTGGATATT | AAAGAAGTCCGAGATATT |
| | (SEQ ID NO: 14) | TTGGA | (SEQ ID NO: 188) |
| | | (SEQ ID NO: 101) | |
| AI309080 | GACCCTTGGAGCAGTGTTGTG | GAGGCTTTATTGACAACGGAGA | AACTTGCCTAGAACTC |
| | (SEQ ID NO: 15) | AG | (SEQ ID NO: 189) |
| | | (SEQ ID NO: 102) | |
| AI341378 | GCCAAAACACTACAAGCCTCTTG | ATCACAAAAATTAGTAAGCCTG | TTTCACCAAAACCC |
| | (SEQ ID NO: 16) | AGATGT | (SEQ ID NO: 190) |
| | | (SEQ ID NO:103) | |
| AI457360 | AGACACTGTCACCCCCTTTCC | CAGCGAACATCTCTGCTTCATC | CCACAAGACTGGCAGAG |
| | (SEQ ID NO: 17) | (SEQ ID NO: 104) | (SEQ ID NO: 191) |
| AI620495 | GCACACTGAGTCTTAGCGTTTCTG | CAACTGGGCTTGGCGTTATT | TGGAAACAGTTTGGATTGTA |
| | (SEQ ID NO: 18) | (SEQ ID NO: 105) | (SEQ ID NO: 192) |
| AI632869 | CTGGAACCAGCTCTCTCCTAATAT | TGACTTGGCAATGTAAGACACA | TTGTGCCCCACACTAAC |
| | TC | CA | (SEQ ID NO: 193) |
| | (SEQ ID NO: 19) | (SEQ ID NO: 106) | |
| AI683181 | CCTGTCAAGATTGCAAGAACATGT | GCTGCTTCGGAACAATATAACGT | AAATGTACGGAGCTTCAT |
| | (SEQ ID NO: 20) | (SEQ ID NO: 107) | (SEQ ID NO: 194) |
| AI685931 | CAAATCCTCCTGCCTGAAGAAG | CTGGTTCTCCCCACAAATGC | TCAGCATCACTTCAGC |
| | (SEQ ID NO: 21) | (SEQ ID NO: 108) | (SEQ ID NO: 195) |
| AI802118 | CCGCTCCTGCAAATTGAGAT | CACACATTGTCTCTAATCCTTA ATGCCTGCCTTTCAA | |
| | (SEQ ID NO: 22) | CAATGAC | (SEQ ID NO: 196) |
| | | (SEQ ID NO: 109) | |
| AI804745 | GGCACCCCGCATTCG | TCCACCCCCCAAAATCAAC | TGTGAGGTTTGTTTGTCC |
| | (SEQ ID NO: 23) | (SEQ ID NO: 110) | (SEQ ID NO: 197) |
| AI952953 | TCACGATGATCCTGACAATGC | CAAAGTGCCCTTCTGCTCCTT | CATGAGAGCCCAGAACA |
| | (SEQ ID NO: 24) | (SEQ ID NO: 111) | (SEQ ID NO: 198) |
| AI985118 | TTTCTAGTGAGCTAACCGTAACA | CACAACGATCTTCTACACGTGA | CCTACAGGATACACGTGAGA |
| | GAGA | CA | (SEQ ID NO: 199) |
| | (SEQ ID NO: 25) | (SEQ ID NO: 112) | |
| AJ000388 | GCCTACCTAGACCAGCAAGCAT | AGTTAAACAGACTGGAAAACAT | CATTTTTAGCTCGCTCATT |
| | (SEQ ID NO: 26) | GGTAAA | (SEQ ID NO: 200) |
| | | (SEQ ID NO: 113) | |
| AK025181 | GCACCGCTGGATGAAAGG | CCTTTGTTTGTTAACTGCTCTT | AGGCTAGAGGCTGAGGG |
| | (SEQ ID NO: 27) | TCC | (SEQ ID NO: 201) |
| | | (SEQ ID NO: 114) | |
| AK027147 | GAGAGGAAGAATTGCAGAGTAGT | CCAAAGAACAGACATGCAGTTA | ATCATGCCAATTCC |
| | TTGT | TTG | (SEQ ID NO: 202) |
| | (SEQ ID NO: 28) | (SEQ ID NO: 115) | |
| AK054605 | CAAGGATTTTTCCAGGCACAGT | ACCTTGGCCTCTCCAAGCA | CATACCTGTAATCCC |
| | (SEQ ID NO: 29) | (SEQ ID NO: 116) | (SEQ ID NO: 203) |
| AL023657 | CCATGTACTGGCAAGACCTGATT | CAGGCCACACTCCACTTTTGT | TATGGATGCCGTGGGAG |
| | (SEQ ID NO: 30) | (SEQ ID NO: 117) | (SEQ ID NO: 204) |
| AL039118 | GCGCAAATGCCGCATAA | GCATATGACCACAGTATCACAA | TTGAGTGATTGTTAATGTTGT |
| | (SEQ ID NO: 31) | TCAA | CT |
| | | (SEQ ID NO: 118) | (SEQ ID NO: 205) |
| AL110274 | CCTCCTGTAGCATGTGTCCAAGT | TCACATTTTTTGTTGCAGTCCAA | AGCCACTAACCAACTAG |
| | (SEQ ID NO: 32) | (SEQ ID NO: 119) | (SEQ ID NO: 206) |
| AL157475 | GTGCTGTTTGCAGTTGTACTCATT | GTTTTACACCCAGCGATGCTT | CTCTCTGCCATCCCC |
| | (SEQ ID NO: 33) | (SEQ ID NO: 120) | (SEQ ID NO: 207) |
| AW118445 | TTCCAGACTTGTCACTGACTTTC | CTGCCCACAGCCTCTTTTTC | CTGGAGCAGGTGGC |
| | CT | (SEQ ID NO: 121) | (SEQ ID NO: 208) |
| | (SEQ ID NO: 34) | | |
| AW194680 | AAGGCGCTGGTGTTTTGCT | AATAACCTGCATTCACCGAAGAG | TGAGTTTTAAGAGATCCC |
| | (SEQ ID NO: 35) | (SEQ ID NO: 122) | (SEQ ID NO: 209) |
| AW291189 | GCCCGGATGAAGCATGAGAT | CCGCTACACGTTGGTGCTA | TTCACGCACTGTCCCTC |
| | (SEQ ID NO: 36) | (SEQ ID NO: 123) | (SEQ ID NO: 210) |
| AW298545 | CCCTTCCCTCAATTTCCTGTTT | AGGAATCTCCGAGTTGAGGAAAA | AAACTGAATGGCACGAAA |
| | (SEQ ID NO: 37) | (SEQ ID NO: 124) | (SEQ ID NO: 211) |
| AW445220 | CACGGGACTGCCACAGA | ACAAGTTTAATGCAACAGGTGA | ATGCTCCGGAAGGCTCA |
| | (SEQ ID NO: 38) | CAAC | (SEQ ID NO: 212) |
| | | (SEQ ID NO: 125) | |
| AW473119 | CAATGCTTTTTGTGCACTACATA | ACAATTTGGCATTTGAGCCTTT | CAGTGTAGAGCTCTTGTTTTA |
| | CTCT | TCC | (SEQ ID NO: 213) |
| | (SEQ ID NO: 39) | (SEQ ID NO: 126) | |
| AY033998 | CACACATACACGAAAGAGAGAGA | AACACTGGCTTATAAAGTCCAT | ACTTTTCAAGGCTTATATTC |
| | AACA | GGT | (SEQ ID NO: 214) |
| | (SEQ ID NO: 40) | (SEQ ID NO: 127) | |
| BC000045 | AAGACACGGCAGCAAGACATC | CAAGTGGGTGTGAGCAGCTTT | CTGCATATTGTTCCAGATAA |
| | (SEQ ID NO: 41) | (SEQ ID NO: 128) | (SEQ ID NO: 215) |
| BC001293 | CATAGCAAAGCAAAGACAGAATGC | AATATCTTTAAATAACACAACT | CCCCCCAAATATT |
| | (SEQ ID NO: 42) | CCCAGACA | (SEQ ID NO: 216) |
| | | (SEQ ID NO: 129) | |
| BC001504 | GTGGAATAGTGGAGGCCTTCAA | GCAGATGCCCTCCAAGATGT | TGATTAGACAAGGCCC |
| | (SEQ ID NO: 43) | (SEQ ID NO: 130) | (SEQ ID NO: 217) |
| BC001639 | GCATGTGTCTGTGTATGTGTGAA | AGGCCCCTTTCCTTCTGAAA | AGAGACACAGCCCTC |
| | TGT | (SEQ ID NO: 131) | (SEQ ID NO: 218) |
| | (SEQ ID NO: 44) | | |
| BC002551 | CCAGGACCATGACAAGGAAAAT | GCCATGCAGGGCCTAGCT | AGCACTTTCCCTTGGTG |
| | (SEQ ID NO: 45) | (SEQ ID NO: 132) | (SEQ ID NO: 219) |
| BC004331 | TGGCGGGGCTTCTGTTTTATTT | TGGCTTTTATTAGCGATTCATG | TAGGCTGGATGCTACCCA |
| | (SEQ ID NO: 46) | AA | (SEQ ID NO: 220) |
| | | (SEQ ID NO: 133) | |
| BC004453 | GATAACTCTGTACGAGGCTTCTC | AGGGAAGCTGCCACAAGTGA | CTAGTGTCTTTTTTTTCTTCAC |
| | TAACC | (SEQ ID NO: 134) | (SEQ ID NO: 221) |
| | (SEQ ID NO: 47) | | |
| BC005364 | AATTCCTCACACCTTGCACCTT | TTTTAAGTACCACTTTTCCTCC | ACTTTTCTGAATTGCTATGACT |
| | (SEQ ID NO: 48) | AACAA | (SEQ ID NO: 222) |
| | | (SEQ ID NO: 135) | |
| BC006537 | AAACCGCCATTGGGCTACT | AGTGTAAGTTCAGTCTGATGGA | CATCAAGGATACAAATCTAC |
| | (SEQ ID NO: 49) | AACC | (SEQ ID NO: 223) |
| | | (SEQ ID NO: 136) | |
| BC006811 | AGAAGACGGAGACAGACATGAGT | CTCAGGACTCTCTGCTAGTACA | CCCGCTCCTGCAGGAG |
| | (SEQ ID NO: 50) | AGT | (SEQ ID NO: 224) |
| | | (SEQ ID NO: 137) | |
| BC006819 | TGCAGAGTGGAAAAGACAAGGAT | TGGCGTCCAGGTCCTTGA | CCGTGGATAAATTG |
| | (SEQ ID NO: 51) | (SEQ ID NO: 138) | (SEQ ID NO: 225) |
| BC008764 | GGGAGAGAGACGGAGCCTTTA | GCCCAAAGGCGTAGAAGGTT | ACAGCTATCTGCTGGCT |
| | (SEQ ID NO: 52) | (SEQ ID NO: 139) | (SEQ ID NO: 226) |
| BC008765 | CTGGGCTGGAATCAGGAATATTT | GGATTAAGTAGAGTTTTGCCAA | CCAAAGAGTGATAGTCTTT |
| | (SEQ ID NO: 53) | AAGC | (SEQ ID NO: 227) |
| | | (SEQ ID NO: 140) | |
| BC009084 | CGATTGTAGCTCTGACATCTGGA | GGGCCCAAAATAGGGAGTGT | TGCACGCTGATGACGC |
| | TT | (SEQ ID NO: 141) | (SEQ ID NO: 228) |
| | (SEQ ID NO: 54) | | |
| BC009237 | TGCCTGGCACAAAGAAGGA | GGCGATGATTGTAAGTTGTTCCA | AAATGATAGTTCGACTCGTCT |
| | (SEQ ID NO: 55) | (SEQ ID NO: 142) | (SEQ ID NO: 229) |
| BC010626 | ACCGAGGAGACTGCTGTGTGA | CATTCAGCAGATGGGCAGACT | CTCCACACTCTTGGGC |
| | (SEQ ID NO: 56) | (SEQ ID NO: 143) | (SEQ ID NO: 230) |
| BC011949 | AAATGCTGCTTTTAAAACATAGG | TGCCTTAACTAGCTCAATTTAT | TAGAATGGTTGAGTGCAAAT |
| | AAA | CTTGTG | (SEQ ID NO: 231) |
| | (SEQ ID NO: 57) | (SEQ ID NO: 144) | |
| BC012926 | GGCCCCGCTGATGCA | TGCTGCAAACTGGGATCCA | ATGGCAGATCTGATACCC |
| | (SEQ ID NO: 58) | (SEQ ID NO: 145) | (SEQ ID NO: 232) |
| BC013117 | GAGCTATTTATCTCTGTTTGTTG | CCACAGTTTTGGCAGTGAACAA | CCAGAGGAATCCCC |
| | GAAAATCC | (SEQ ID NO: 146) | (SEQ ID NO: 233) |
| | (SEQ ID NO: 59) | | |
| BC015754 | CATTTTGATCTGTAACTGCACAA | CAAGATGGATCCACTACTTTAC | CTGCAGCAAACCCCA |
| | CCC | ATGGA | (SEQ ID NO: 234) |
| | (SEQ ID NO: 60) | (SEQ ID NO: 147) | |
| BC017586 | CCATGTGGCTCCAAATGACTAA | TTAGGATGAGTGTGAAATCAAA | TGTCAGCTCAAAAACCAGA |
| | (SEQ ID NO: 61) | TACGA | (SEQ ID NO: 235) |
| | | (SEQ ID NO: 148) | |
| BE552004 | AGGCCCAGGTTTCGACAGA | GGCTCCGAAATGGCATCTC | AGGGAGAGAAAACC |
| | (SEQ ID NO: 62) | (SEQ ID NO: 149) | (SEQ ID NO: 236) |
| BE962007 | GTGAGAAACTGAATGTATTATTC | GTGCAAATTGACTTTTACATTC | ACTGAGTGCCTTCATTT |
| | AGGAAGA | AACTTTAG | (SEQ ID NO: 237) |
| | (SEQ ID NO: 63) | (SEQ ID NO: 150) | |
| BF224381 | ACGCCACAGGAGGACATGTT | TCACACCCCCATACTCTTCTGTT | CTGCAGATGTAGTTGCC |
| | (SEQ ID NO: 64) | (SEQ ID NO: 151) | (SEQ ID NO: 238) |
| BF437393 | CGCTGTGGGCAATTGTTACA | CCCATAAAGCAATTCACGGATA | TTCACAGTAAACCTAAGAACA |
| | (SEQ ID NO: 65) | CAG | CT |
| | | (SEQ ID NO: 152) | (SEQ ID NO: 239) |
| BF446419 | AGCTCCACAACCCTGTTTGG | GCTTGGGAAACCGCACTTT | ACTGCAGGACCAGAAG |
| | (SEQ ID NO: 66) | (SEQ ID NO: 153) | (SEQ ID NO: 240) |
| BF592799 | GCCATGACTGGTGATTTCATGA | ATGCATGGGCCATTGATCTT | CCTCCGTAGGCATCA |
| | (SEQ ID NO: 67) | (SEQ ID NO: 154) | (SEQ ID NO: 241) |
| BI493248 | AAATGTGTAGTTTCTTAATCGCA | GGTCACATAAAAATACATGAGG | TGCAACACTGTGTATTAG |
| | CTACCT | ATGATAA | (SEQ ID NO: 242) |
| | (SEQ ID NO: 68) | (SEQ ID NO: 155) | |
| H05388 | ACAGGTTCTTATCTGCAAGGTTC | TGACTGGCCCTGCAGAATACT | TTGCTTAGAOATTGTTTTC |
| | AA | (SEQ ID NO: 156) | (SEQ ID NO: 243) |
| | (SEQ ID NO: 69) | | |
| H07885 | GTCACTGTCATAGCAGCTGTGAT | CCOACTCCCCATCAACCA | CAAGGAAGGGTGCTGCA |
| | TT | (SEQ ID NO: 157) | (SEQ ID NO: 244) |
| | (SEQ ID NO: 70) | | |
| H09748 | TGTACAAGATTTTGGGCCTCTTTT | AAATGGACAGACACATGCTGAA | TCCTTAATGTCACAATGTT |
| | (SEQ ID NO: 71) | CT | (SEQ ID NO: 245) |
| | | (SEQ ID NO: 158) | |
| M95585 | TTGTAACATGGACCATCCAAATT | CCAAGAGAGACCAGTGCTCAAA | CAAATGGTAGCTGAAAAA |
| | TAT | TA | (SEQ ID NO: 246) |
| | (SEQ ID NO: 72) | (SEQ ID NO: 159) | |
| N64339 | GCTTTCTGAATGTAGACGGAACA | TTGGCAAACGGATGAGTTAAAAA | TGGAAGCAGAAGGC |
| | GT | (SEQ ID NO: 160) | (SEQ ID NO: 247) |
| | (SEQ ID NO: 73) | | |
| NM_000065 | TCTTCAATGAGTTAATAAACAGA | TGAATGAAGATATGAAAGCTGG | CCTCTGAAACACATTCTTG |
| | AATCTCCAGAA | GCTT | (SEQ ID NO: 248) |
| | (SEQ ID NO: 74) | (SEQ ID NO: 161) | |
| NM_001337 | GTTAGACCACAAATAGTGCTCGCT | ATGAATACACAGTCTGGTAGAG | TTCTATGTAGTTTGGTAATTA |
| | (SEQ ID NO: 75) | TCTTCT | TCA |
| | | (SEQ ID NO: 162) | (SEQ ID NO: 249) |
| NM_003914 | TTCCAGAACTTCACCTCCATATCA | GATCCAACGTGCAGAAGCCTAT | AGTGCCAATAATCG |
| | (SEQ ID NO: 76) | (SEQ ID NO: 163) | (SEQ ID NO: 250) |
| NM_004062 | GCCTGGACACCAACTTTATGG | GGGCTTTATTATTGGGCAAACA | AGTGCTCCAAATGTC |
| | (SEQ ID NO: 77) | (SEQ ID NO: 164) | (SEQ ID NO: 251) |
| NM_004063 | CAAACACAACCTACTCTGCAAACC | GCATGGCAGGTAGTGAGGAAA | AAAGGAACCAGTCAGCTG |
| | (SEQ ID NO: 78) | (SEQ ID NO: 165) | (SEQ ID NO: 252) |
| NM_004496 | CATTGCCATCGTGTGCTTGT | ACCCTCTGGCTATACTAACACC | CAGTGTTATGCACTTTC |
| | (SEQ ID NO: 79) | AACT | (SEQ ID NO: 253) |
| | | (SEQ ID NO: 166) | |
| NM_006115 | GATTCTGGCTTGGGAAGTACATG | GCTTCTCTTTATTTTCAACAGT | AATCCCTGTGTAGACTGT |
| | (SEQ ID NO: 80) | TTCTTTAC | (SEQ ID NO: 254) |
| | | (SEQ ID NO: 167) | |
| NM_019894 | CCCACACTACTGAATGGAAGCA | CCTCTCCAGCCCACAGTGAT | CTGTCTTGTAAAAGCC |
| | (SEQ ID NO: 81) | (SEQ ID NO: 168) | (SEQ ID NO: 255) |
| NM_033229 | GCGTGAGGCGAGAGAACAG | GAGCTGAGGGCCTAAGATAAAT | AGTCTCGAACAGCGGTT |
| | (SEQ ID NO: 82) | AAAGT | (SEQ ID NO: 256) |
| | | (SEQ ID NO: 169) | |
| R15881 | TCAGAACCCACTTTCAAGATGCT | GCTGCTTGCGCCTCTTTTT | TGCTGTGCCAGTGTGA |
| | (SEQ ID NO: 83) | (SEQ ID NO: 170) | (SEQ ID NO: 257) |
| R45389 | AGTGGATCAGACAGTACGACTTT | TCCAAAGCAGCTTAGGTGAAAAA | CTGGTGAATGTAAACAAT |
| | GA | (SEQ ID NO: 171) | (SEQ ID NO: 258) |
| | (SEQ ID NO: 84) | | |
| R61469 | TTCCCCGGGCATTTGTT | CATGTCGCAGGGTTAAGTATGA | TTCAAACAGACTTTAACCTC |
| | (SEQ ID NO: 85) | TG | (SEQ ID NO: 259) |
| | | (SEQ ID NO: 172) | |
| X69699 | TGTTTGGGTCAAGCTTCCTTCT | GGCAAAGAGAGACATTTCACTC | CCCCCAGACTTTGG |
| | (SEQ ID NO: 86) | AGA | (SEQ ID NO: 260) |
| | | (SEQ ID NO: 169) | |
| X96757 | CCCTGCCTCTCAGAGGGTTT | ATTCCAAGGCCCCCTTAAGA | CTCTCCCAATTTTC |
| | (SEQ ID NO: 87) | (SEQ ID NO: 174) | (SEQ ID NO: 261) |

What is claimed is:

1. A method for diagnosis of a disease or condition in an individual, said method comprising:
a) providing a primary self organizing map (SOM) constructed using a plurality of data sets of measurements obtained from a plurality of individuals each having a disease or condition;
b) preparing a secondary SOM using a distinct labeling set, said distinct labeling set encompassing data sets of measurements of a particular disease or condition, said secondary SOM including a sample data set obtained from a sample of said individual; and
c) preparing a result from said secondary SOM that reveals the extent of similarity between the data sets of measurements of the distinct labeling set and said sample data set of said individual;
whereby a medical practitioner can use said result to diagnose said disease or condition.

2. The method of claim 1, wherein in step a) said plurality of individuals represents a plurality of diseases or conditions.

3. The method of claim 2, wherein step b) is repeated to prepare multiple secondary SOMs for different diseases or conditions.

4. The method of claim 3, wherein said result is a display of one or more of said multiple secondary SOMs.

5. The method of claim 1, wherein said result is a display of said sample data set with respect to said data sets of measurements of said distinct labeling set.

6. The method of claim 1, wherein said result is a probability that said sample data set is similar to one or more of said data sets of measurements of said distinct labeling set.

7. The method of claim 1, wherein said data sets comprise gene expression levels or protein levels.

8. The method of claim 7, wherein said data sets comprise gene expression levels.

9. The method of claim 1, wherein each of said plurality of different diseases or conditions is a cancer.

10. The method of claim 9, wherein said cancer is selected from the group consisting of tumors of type adrenal, brain, breast, carcinoid-intestine, cervix-adeno, cervix-squamous, endometrium, gallbladder, germ-cell-ovary, gastrointestinal stromal, kidney, leiomyosarcoma, liver, lung-adeno-large cell, lung-small cell, lung-squamous, lymphoma-B cell, lymphoma-Hodgkin, lymphoma-T cell, memigioma, mesothelioma, osteosarcoma, ovary-clear, ovary-serous, pancreas, skin-basal cell, skin-melanoma, skin-squamous, small bowel, large bowel, soft tissue-liposarcoma, soft tissue-malignant fibrous histiocytoma, soft tissue-sarcoma-synovial, stomach-adeno, testis-other, testis-seminoma, thyroid-follicular-papillary, thyroid-medullary, and urinary bladder.

11. The method of claim 9, wherein said cancer is selected from the group consisting of melanoma, pancreatic cancer, colorectal cancer, non-small cell lung cancer, breast cancer, small cell lung cancer, ovarian cancer, prostate cancer, stomach cancer, and kidney cancer.

12. The method of claim 1, wherein said sample data set and said data sets each comprise a data vector of continuous or discrete scalars.

13. The method of claim 12, wherein the dimensionality of said data vector of scalars is greater than 2.

14. The method of claim 12, wherein the dimensionality of said data vector of scalars is greater than 20.

15. The method of claim 12, wherein the dimensionality of said data vector of scalars is at least 29.

16. The method of claim 1, further comprising displaying annotation associated with a map cell of said primary or said secondary SOM.

17. The method of claim 16, wherein said annotation is displayed after said map cell is picked.

18. The method of claim 17, further comprising displaying annotation associated with a map cell near said picked map cell.

19. The method of claim 1, wherein said medical practitioner is a non-veterinary medical practitioner.

20. The method of claim 1, wherein said individual presents with cancer of unknown primary.

21. The method of claim 1, wherein said diagnosis is the primary site of a metastatic cancer.

22. The method of claim 1, wherein said result is a probability P_{related}ⁱ that said sample data set is related to one of said different diseases or conditions.

23. The method of claim 22, wherein the calculation of said probability P_{related}ⁱ comprises the steps of:
i) determining a plurality of nearest neighbors of said sample data set with respect to said data sets of measurements representing a plurality of different diseases or conditions; and
ii) determining if said plurality of nearest neighbors individually represent the same disease or condition.

24. The method of claim 23, when each of said plurality of nearest neighbors represents the same disease or condition, wherein P_{related}ⁱ=1.0.

25. The method of claim 23, when each of said plurality of nearest neighbors do not all represent the same disease or condition, further comprising the steps of:
iii) calculating a probability factor P_{cluster}ⁱ for one or more of said diseases or conditions represented in said plurality of nearest neighbors, wherein P_{related}ⁱ=P_{cluster}ⁱ.

26. The method of claim 25, wherein said probability factor P_{cluster}ⁱ is calculated
by evaluating the expression
$$\frac{\frac{1}{{d}_{j}^{2}}}{{\sum }_{p = 1}^{T} \frac{1}{{d}_{p}^{2}}}$$
for one or more of said disease or condition represented in said plurality of nearest neighbors,
wherein:
dⱼ is the Euclidian distance between said sample data set and the closest cluster center of T clusters obtaining from a clustering of said distinct labeling sets representing said disease or conditions represented in said plurality of nearest neighbors; and
dₚ is the Euclidian distance between said sample data set and any of said T cluster centers;

27. The method of claim 23, when each of said plurality of nearest neighbors do not all represent the same disease or condition, further comprising the steps of:
iii) calculating a probability factor Pₜᵢₛₛᵤₑⁱ for one or more of said diseases or conditions represented in said plurality of nearest neighbors, wherein P_{related}ⁱ=Pₜᵢₛₛᵤₑⁱ.

28. The method of claim 27, wherein said probability factor Pₜᵢₛₛᵤₑⁱ is calculated by evaluating the expression
$$\frac{\frac{1}{{d}_{k}^{2}}}{{\sum }_{q = 1}^{U} \frac{1}{{d}_{q}^{2}}}$$
for one or more of said diseases or conditions represented in said plurality of nearest neighbors, wherein:
dₖ is the Euclidian distance between said sample data set and the center of said distinct labeling set representing said disease or condition; and
d_{q} is the Euclidian distance between said sample data set and any of U centers of said distinct labeling set representing said disease or condition.

29. The method of claim 23, when each of said plurality of nearest neighbors do not all represent the same disease or condition, further comprising the steps of:
iii) calculating a probability factor P_{cluster}ⁱ for one or more of said diseases or conditions represented in said plurality of nearest neighbors.
iv) calculating a probability factor Pₜᵢₛₛᵤₑⁱ for one or more of said diseases or conditions represented in said plurality of nearest neighbors; and
v) calculating probability P_{related}ⁱ=αP_{cluster}ⁱ+βPₜᵢₛₛᵤₑ wherein α+β=1.

30. The method of claim 29, wherein α0.3 and β=0.7.

31. A method for constructing a self-organizing map (SOM) useful in the diagnosis of an individual suffering from a disease or condition, said method comprising:
a) constructing a primary self organizing map (SOM) by using a plurality of data sets of measurements, said data sets representing a plurality of different diseases or conditions, said data sets obtained from a plurality of individuals each having a disease or condition; and
b) forming at least one secondary SOM using at least one distinct labeling set, said distinct labeling set encompassing data sets of measurements of a particular disease or condition, said secondary SOM including a sample data set obtained from a sample of said individual,
thereby providing a SOM suitable for diagnosis of a disease or condition in said individual.

32. The method of claim 31, wherein said sample data set and said data sets each comprise a data vector continuous or discrete scalars.

33. The method of claim 32, wherein the dimensionality of said data vector of scalars is greater than 2.

34. The method of claim 32, wherein the dimensionality of said data vector of scalars is at least 29.

35. The method of claim 31, wherein step b) is repeated to prepare multiple secondary SOMs for different diseases or conditions.

36. A method of displaying a self organizing map (SOM) useful in the diagnosis of an individual suffering from a disease or condition, said method comprising:
a) constructing a primary self organizing map (SOM) by using a plurality of data sets of measurements, said data sets representing a plurality of different diseases or conditions, said data sets obtained from a plurality of individuals each having a disease or condition;
b) forming at least one secondary SOM using at least one distinct labeling set, said distinct labeling set encompassing data sets of measurements of a particular disease or condition, said secondary SOM including a sample data set obtained from a sample of said individual; and
c) displaying said primary SOM or said at least one secondary SOM.

37. The method of claim 36, further comprising displaying annotation associated with a map cell of said primary or said secondary SOM.

38. The method of claim 37, wherein said annotation is displayed after said map cell is picked.

39. The method of claim 38, further comprising displaying annotation associated with a map cell near said picked map cell.

40. A program product comprising machine-readable program code for causing a machine to perform the following method steps:
a) constructing a primary self organizing map (SOM) using a plurality of data sets of measurements obtained from a plurality of individuals each having a disease or condition; and
b) preparing a secondary SOM using at least one distinct labeling set, said distinct labeling set encompassing data sets of measurements of a particular disease or condition, said secondary SOM including a sample data set obtained from a sample of said individual.

41. The program product of claim 40, further comprising machine-readable program code for causing a machine to perform the following method step:
c) preparing a result from said secondary SOM that reveals the extent of similarity between the data sets of measurements of the distinct labeling set and said sample data set of said individual.

42. The program product of claim 41, wherein said result is a probability P_{related}ⁱ that said sample data set is related to one of said different diseases or conditions.

43. The program product of claim 42, further comprising machine-readable program code for causing a machine to display said probability P_{related}ⁱ.

44. The program product of claim 40, further comprising machine-readable program code for causing a machine to display said primary SOM or said secondary SOM.

45. The program product of claim 40, further comprising machine-readable program code for causing a machine to display annotation associated with a map cell of said primary or secondary SOM.

46. The program product of claim 45, wherein said annotation is displayed after said map cell is picked.

47. The method of claim 46, further comprising machine-readable program code for causing a machine to display annotation associated with map cells near said picked map cell.

48. A method for providing therapy response information associated with at least one pickable map cell of a primary or secondary SOM, said method comprising:
a) providing annotation of therapy response information for said at least one pickable map cell of a primary or secondary SOM, and
b) displaying said annotation of therapy response information after said map cell is picked.

49. The method of claim 48, wherein said primary SOM is constructed using a plurality of data sets of measurements obtained from a plurality of individuals each having a disease or condition, and said secondary SOM is prepared using a distinct labeling set, said distinct labeling set encompassing data sets of measurements of a particular disease or condition, said secondary SOM including a sample data set obtained from a sample of said individual.

50. The method of claim 48, further comprising displaying therapy response information of map cells near said picked map cell.

51. A method for reducing the number of biological markers required to construct a primary SOM useful for the diagnosis of an individual having a disease or condition, said method comprising using a reduction method to find the minimum set of biological markers that contribute to a model to predict said possible diseases or conditions, said method selected from the group consisting of forward stepwise logistic regression, backward stepwise logistic regression, linear regression, logistic regression, and non-stepwise logistic regression,

52. The method of claims 51, wherein said disease or condition is cancer of unknown primary.

53. A method for diagnosis of cancer of unknown primary in an individual, said method comprising:
a) providing a primary self organizing map (SOM) constructed using a plurality of data sets of measurements obtained from a plurality of individuals representing a plurality of particular cancers;
b) preparing a plurality of secondary SOMs each with a distinct labeling set, each of said distinct labeling sets encompassing data sets of measurements obtained from individuals having a particular cancer, said secondary SOM including a sample data set obtained from a sample of said individual;
c) preparing a result from said plurality of secondary SOMs that reveals the extent of similarity between the data sets of measurements of the distinct labeling set and said sample data set of said individual; and
d) providing said result to a medical practitioner for use to diagnosis said cancer of unknown primary, wherein said result is selected from the group consisting of said primary SOM, one or more of said secondary SOMs, a display of said primary SOM, a display of said one or more of said secondary SOMs, and a probability that said sample data set is one or more of said particular cancers.


SEQUENCE LISTING (261 sequences)

SEQ ID NO: 1 - DNA, 23 bp, Artificial Sequence
  Description of Artificial Sequence Synthetic primer
cagtctagacatgctgcaaggaa

SEQ ID NO: 2 - DNA, 18 bp, Artificial Sequence
  Description of Artificial Sequence Synthetic primer
cctggagacccggagaca

SEQ ID NO: 3 - DNA, 26 bp, Artificial Sequence
  Description of Artificial Sequence Synthetic primer
ttgtactgagctgtgaagtcagtgtt

SEQ ID NO: 4 - DNA, 21 bp, Artificial Sequence
  Description of Artificial Sequence Synthetic primer
ccgcggtgtacaatacccata

SEQ ID NO: 5 - DNA, 26 bp, Artificial Sequence
  Description of Artificial Sequence Synthetic primer
cccttacattctgcacttcatagttg

SEQ ID NO: 6 - DNA, 17 bp, Artificial Sequence
  Description of Artificial Sequence Synthetic primer
ggcggagcgagagcaaa

SEQ ID NO: 7 - DNA, 23 bp, Artificial Sequence
  Description of Artificial Sequence Synthetic primer
tgtgcctcctcttagcatctgtt

SEQ ID NO: 8 - DNA, 25 bp, Artificial Sequence
  Description of Artificial Sequence Synthetic primer
gagaagattgtctaccacagcaagt

SEQ ID NO: 9 - DNA, 19 bp, Artificial Sequence
  Description of Artificial Sequence Synthetic primer
ccagcggtttccacttgtg

SEQ ID NO: 10 - DNA, 20 bp, Artificial Sequence
  Description of Artificial Sequence Synthetic primer
tcaagtggccgaagccttac

SEQ ID NO: 11 - DNA, 23 bp, Artificial Sequence
  Description of Artificial Sequence Synthetic primer
ggcaagttttcaagcactgagtt

SEQ ID NO: 12 - DNA, 24 bp, Artificial Sequence
  Description of Artificial Sequence Synthetic primer
cattctcaacagggaaaccctact

SEQ ID NO: 13 - DNA, 19 bp, Artificial Sequence
  Description of Artificial Sequence Synthetic primer
agaccatcgccagcatctg

SEQ ID NO: 14 - DNA, 24 bp, Artificial Sequence
  Description of Artificial Sequence Synthetic primer
tgaacaagatgaaccaatgtggat

SEQ ID NO: 15 - DNA, 21 bp, Artificial Sequence
  Description of Artificial Sequence Synthetic primer
gacccttggagcagtgttgtg

SEQ ID NO: 16 - DNA, 23 bp, Artificial Sequence
  Description of Artificial Sequence Synthetic primer
gccaaaacactacaagcctcttg

SEQ ID NO: 17 - DNA, 21 bp, Artificial Sequence
  Description of Artificial Sequence Synthetic primer
agacactgtcaccccctttcc

SEQ ID NO: 18 - DNA, 24 bp, Artificial Sequence
  Description of Artificial Sequence Synthetic primer
gcacactgagtcttagcgtttctg

SEQ ID NO: 19 - DNA, 26 bp, Artificial Sequence
  Description of Artificial Sequence Synthetic primer
ctggaaccagctctctcctaatattc

SEQ ID NO: 20 - DNA, 24 bp, Artificial Sequence
  Description of Artificial Sequence Synthetic primer
cctgtcaagattgcaagaacatgt

SEQ ID NO: 21 - DNA, 22 bp, Artificial Sequence
  Description of Artificial Sequence Synthetic primer
caaatcctcctgcctgaagaag

SEQ ID NO: 22 - DNA, 20 bp, Artificial Sequence
  Description of Artificial Sequence Synthetic primer
ccgctcctgcaaattgagat

SEQ ID NO: 23 - DNA, 15 bp, Artificial Sequence
  Description of Artificial Sequence Synthetic primer
ggcaccccgcattcg

SEQ ID NO: 24 - DNA, 21 bp, Artificial Sequence
  Description of Artificial Sequence Synthetic primer
tcacgatgatcctgacaatgc

SEQ ID NO: 25 - DNA, 27 bp, Artificial Sequence
  Description of Artificial Sequence Synthetic primer
tttctagtgagctaaccgtaacagaga

SEQ ID NO: 26 - DNA, 22 bp, Artificial Sequence
  Description of Artificial Sequence Synthetic primer
gcctacctagaccagcaagcat

SEQ ID NO: 27 - DNA, 18 bp, Artificial Sequence
  Description of Artificial Sequence Synthetic primer
gcaccgctggatgaaagg

SEQ ID NO: 28 - DNA, 27 bp, Artificial Sequence
  Description of Artificial Sequence Synthetic primer
gagaggaagaattgcagagtagtttgt

SEQ ID NO: 29 - DNA, 22 bp, Artificial Sequence
  Description of Artificial Sequence Synthetic primer
caaggatttttccaggcacagt

SEQ ID NO: 30 - DNA, 23 bp, Artificial Sequence
  Description of Artificial Sequence Synthetic primer
ccatgtactggcaagacctgatt

SEQ ID NO: 31 - DNA, 17 bp, Artificial Sequence
  Description of Artificial Sequence Synthetic primer
gcgcaaatgccgcataa

SEQ ID NO: 32 - DNA, 23 bp, Artificial Sequence
  Description of Artificial Sequence Synthetic primer
cctcctgtagcatgtgtccaagt

SEQ ID NO: 33 - DNA, 24 bp, Artificial Sequence
  Description of Artificial Sequence Synthetic primer
gtgctgtttgcagttgtactcatt

SEQ ID NO: 34 - DNA, 25 bp, Artificial Sequence
  Description of Artificial Sequence Synthetic primer
ttccagacttgtcactgactttcct

SEQ ID NO: 35 - DNA, 19 bp, Artificial Sequence
  Description of Artificial Sequence Synthetic primer
aaggcgctggtgttttgct

SEQ ID NO: 36 - DNA, 20 bp, Artificial Sequence
  Description of Artificial Sequence Synthetic primer
gcccggatgaagcatgagat

SEQ ID NO: 37 - DNA, 22 bp, Artificial Sequence
  Description of Artificial Sequence Synthetic primer
cccttccctcaatttcctgttt

SEQ ID NO: 38 - DNA, 17 bp, Artificial Sequence
  Description of Artificial Sequence Synthetic primer
cacgggactgccacaga

SEQ ID NO: 39 - DNA, 27 bp, Artificial Sequence
  Description of Artificial Sequence Synthetic primer
caatgctttttgtgcactacatactct

SEQ ID NO: 40 - DNA, 27 bp, Artificial Sequence
  Description of Artificial Sequence Synthetic primer
cacacatacacgaaagagagagaaaca

SEQ ID NO: 41 - DNA, 21 bp, Artificial Sequence
  Description of Artificial Sequence Synthetic primer
aagacacggcagcaagacatc

SEQ ID NO: 42 - DNA, 24 bp, Artificial Sequence
  Description of Artificial Sequence Synthetic primer
catagcaaagcaaagacagaatgc

SEQ ID NO: 43 - DNA, 22 bp, Artificial Sequence
  Description of Artificial Sequence Synthetic primer
gtggaatagtggaggccttcaa

SEQ ID NO: 44 - DNA, 26 bp, Artificial Sequence
  Description of Artificial Sequence Synthetic primer
gcatgtgtctgtgtatgtgtgaatgt

SEQ ID NO: 45 - DNA, 22 bp, Artificial Sequence
  Description of Artificial Sequence Synthetic primer
ccaggaccatgacaaggaaaat

SEQ ID NO: 46 - DNA, 22 bp, Artificial Sequence
  Description of Artificial Sequence Synthetic primer
tggcggggcttctgttttattt

SEQ ID NO: 47 - DNA, 28 bp, Artificial Sequence
  Description of Artificial Sequence Synthetic primer
gataactctgtacgaggcttctctaacc

SEQ ID NO: 48 - DNA, 22 bp, Artificial Sequence
  Description of Artificial Sequence Synthetic primer
aattcctcacaccttgcacctt

SEQ ID NO: 49 - DNA, 19 bp, Artificial Sequence
  Description of Artificial Sequence Synthetic primer
aaaccgccattgggctact

SEQ ID NO: 50 - DNA, 23 bp, Artificial Sequence
  Description of Artificial Sequence Synthetic primer
agaagacggagacagacatgagt

SEQ ID NO: 51 - DNA, 23 bp, Artificial Sequence
  Description of Artificial Sequence Synthetic primer
tgcagagtggaaaagacaaggat

SEQ ID NO: 52 - DNA, 21 bp, Artificial Sequence
  Description of Artificial Sequence Synthetic primer
gggagagagacggagccttta

SEQ ID NO: 53 - DNA, 23 bp, Artificial Sequence
  Description of Artificial Sequence Synthetic primer
ctgggctggaatcaggaatattt

SEQ ID NO: 54 - DNA, 25 bp, Artificial Sequence
  Description of Artificial Sequence Synthetic primer
cgattgtagctctgacatctggatt

SEQ ID NO: 55 - DNA, 19 bp, Artificial Sequence
  Description of Artificial Sequence Synthetic primer
tgcctggcacaaagaagga

SEQ ID NO: 56 - DNA, 21 bp, Artificial Sequence
  Description of Artificial Sequence Synthetic primer
acccaggagactgctgtgtga

SEQ ID NO: 57 - DNA, 26 bp, Artificial Sequence
  Description of Artificial Sequence Synthetic primer
aaatgctgcttttaaaacataggaaa

SEQ ID NO: 58 - DNA, 15 bp, Artificial Sequence
  Description of Artificial Sequence Synthetic primer
ggccccgctgatgca

SEQ ID NO: 59 - DNA, 31 bp, Artificial Sequence
  Description of Artificial Sequence Synthetic primer
gagctatttatctctgtttgttggaaaatcc

SEQ ID NO: 60 - DNA, 26 bp, Artificial Sequence
  Description of Artificial Sequence Synthetic primer
cattttgatctgtaactgcacaaccc

SEQ ID NO: 61 - DNA, 22 bp, Artificial Sequence
  Description of Artificial Sequence Synthetic primer
ccatgtggctccaaatgactaa

SEQ ID NO: 62 - DNA, 19 bp, Artificial Sequence
  Description of Artificial Sequence Synthetic primer
aggcccaggtttcgacaga

SEQ ID NO: 63 - DNA, 30 bp, Artificial Sequence
  Description of Artificial Sequence Synthetic primer
gtgagaaactgaatgtattattcaggaaga

SEQ ID NO: 64 - DNA, 20 bp, Artificial Sequence
  Description of Artificial Sequence Synthetic primer
acgccacaggaggacatgtt

SEQ ID NO: 65 - DNA, 20 bp, Artificial Sequence
  Description of Artificial Sequence Synthetic primer
cgctgtgggcaattgttaca

SEQ ID NO: 66 - DNA, 20 bp, Artificial Sequence
  Description of Artificial Sequence Synthetic primer
agctccacaaccctgtttgg

SEQ ID NO: 67 - DNA, 22 bp, Artificial Sequence
  Description of Artificial Sequence Synthetic primer
gccatgactggtgatttcatga

SEQ ID NO: 68 - DNA, 29 bp, Artificial Sequence
  Description of Artificial Sequence Synthetic primer
aaatgtgtagtttcttaatcgcactacct

SEQ ID NO: 69 - DNA, 25 bp, Artificial Sequence
  Description of Artificial Sequence Synthetic primer
acaggttcttatctgcaaggttcaa

SEQ ID NO: 70 - DNA, 25 bp, Artificial Sequence
  Description of Artificial Sequence Synthetic primer
gtcactgtcatagcagctgtgattt

SEQ ID NO: 71 - DNA, 24 bp, Artificial Sequence
  Description of Artificial Sequence Synthetic primer
tgtacaagattttgggcctctttt

SEQ ID NO: 72 - DNA, 26 bp, Artificial Sequence
  Description of Artificial Sequence Synthetic primer
ttgtaacatggaccatccaaatttat

SEQ ID NO: 73 - DNA, 25 bp, Artificial Sequence
  Description of Artificial Sequence Synthetic primer
gctttctgaatgtagacggaacagt

SEQ ID NO: 74 - DNA, 34 bp, Artificial Sequence
  Description of Artificial Sequence Synthetic primer
tcttcaatgagttaataaacagaaatctccagaa

SEQ ID NO: 75 - DNA, 24 bp, Artificial Sequence
  Description of Artificial Sequence Synthetic primer
gttagaccacaaatagtgctcgct

SEQ ID NO: 76 - DNA, 24 bp, Artificial Sequence
  Description of Artificial Sequence Synthetic primer
ttccagaacttcacctccatatca

SEQ ID NO: 77 - DNA, 21 bp, Artificial Sequence
  Description of Artificial Sequence Synthetic primer
gcctggacaccaactttatgg

SEQ ID NO: 78 - DNA, 24 bp, Artificial Sequence
  Description of Artificial Sequence Synthetic primer
caaacacaacctactctgcaaacc

SEQ ID NO: 79 - DNA, 20 bp, Artificial Sequence
  Description of Artificial Sequence Synthetic primer
cattgccatcgtgtgcttgt

SEQ ID NO: 80 - DNA, 23 bp, Artificial Sequence
  Description of Artificial Sequence Synthetic primer
gattctggcttgggaagtacatg

SEQ ID NO: 81 - DNA, 22 bp, Artificial Sequence
  Description of Artificial Sequence Synthetic primer
cccacactactgaatggaagca

SEQ ID NO: 82 - DNA, 19 bp, Artificial Sequence
  Description of Artificial Sequence Synthetic primer
gcgtgaggcgagagaacag

SEQ ID NO: 83 - DNA, 23 bp, Artificial Sequence
  Description of Artificial Sequence Synthetic primer
tcagaacccactttcaagatgct

SEQ ID NO: 84 - DNA, 25 bp, Artificial Sequence
  Description of Artificial Sequence Synthetic primer
agtggatcagacagtacgactttga

SEQ ID NO: 85 - DNA, 17 bp, Artificial Sequence
  Description of Artificial Sequence Synthetic primer
ttccccgggcatttgtt

SEQ ID NO: 86 - DNA, 22 bp, Artificial Sequence
  Description of Artificial Sequence Synthetic primer
tgtttgggtcaagcttccttct

SEQ ID NO: 87 - DNA, 20 bp, Artificial Sequence
  Description of Artificial Sequence Synthetic primer
ccctgcctctcagagggttt

SEQ ID NO: 88 - DNA, 25 bp, Artificial Sequence
  Description of Artificial Sequence Synthetic primer
tgtgcgttcaagaaaggatatggaa

SEQ ID NO: 89 - DNA, 21 bp, Artificial Sequence
  Description of Artificial Sequence Synthetic primer
agtcgtgacagttcccgtgtt

SEQ ID NO: 90 - DNA, 20 bp, Artificial Sequence
  Description of Artificial Sequence Synthetic primer
gccaccatccaaacctcaat

SEQ ID NO: 91 - DNA, 22 bp, Artificial Sequence
  Description of Artificial Sequence Synthetic primer
ggaagtaaaagcagccagcaat

SEQ ID NO: 92 - DNA, 20 bp, Artificial Sequence
  Description of Artificial Sequence Synthetic primer
ccctttccaagtccctccat

SEQ ID NO: 93 - DNA, 26 bp, Artificial Sequence
  Description of Artificial Sequence Synthetic primer
ctgatcagaaatgaaaagcgtgtctt

SEQ ID NO: 94 - DNA, 23 bp, Artificial Sequence
  Description of Artificial Sequence Synthetic primer
ggcaggcattttattcatcattt

SEQ ID NO: 95 - DNA, 22 bp, Artificial Sequence
  Description of Artificial Sequence Synthetic primer
cagcttcataagggcgatgtca

SEQ ID NO: 96 - DNA, 23 bp, Artificial Sequence
  Description of Artificial Sequence Synthetic primer
cacaacgactgaaaatgcacttg

SEQ ID NO: 97 - DNA, 21 bp, Artificial Sequence
  Description of Artificial Sequence Synthetic primer
ggctcagggtttgaactcgat

SEQ ID NO: 98 - DNA, 27 bp, Artificial Sequence
  Description of Artificial Sequence Synthetic primer
acattaaggaagcatttgtcactctct

SEQ ID NO: 99 - DNA, 31 bp, Artificial Sequence
  Description of Artificial Sequence Synthetic primer
tcccatgattcttcaaaaagttctgtatctt

SEQ ID NO: 100 - DNA, 23 bp, Artificial Sequence
  Description of Artificial Sequence Synthetic primer
tgcctttgctgtggtaagaattc

SEQ ID NO: 101 - DNA, 27 bp, Artificial Sequence
  Description of Artificial Sequence Synthetic primer
cctttaacaatgtctggatattttgga

SEQ ID NO: 102 - DNA, 24 bp, Artificial Sequence
  Description of Artificial Sequence Synthetic primer
gaggctttattgacaacggagaag

SEQ ID NO: 103 - DNA, 28 bp, Artificial Sequence
  Description of Artificial Sequence Synthetic primer
atcacaaaaattagtaagcctgagatgt

SEQ ID NO: 104 - DNA, 22 bp, Artificial Sequence
  Description of Artificial Sequence Synthetic primer
cagcgaacatctctgcttcatc

SEQ ID NO: 105 - DNA, 20 bp, Artificial Sequence
  Description of Artificial Sequence Synthetic primer
caactgggcttggcgttatt

SEQ ID NO: 106 - DNA, 24 bp, Artificial Sequence
  Description of Artificial Sequence Synthetic primer
tgacttggcaatgtaagacacaca

SEQ ID NO: 107 - DNA, 23 bp, Artificial Sequence
  Description of Artificial Sequence Synthetic primer
gctgcttcggaacaatataacgt

SEQ ID NO: 108 - DNA, 20 bp, Artificial Sequence
  Description of Artificial Sequence Synthetic primer
ctggttctccccacaaatgc

SEQ ID NO: 109 - DNA, 29 bp, Artificial Sequence
  Description of Artificial Sequence Synthetic primer
cacacattgtctctaatccttacaatgac

SEQ ID NO: 110 - DNA, 19 bp, Artificial Sequence
  Description of Artificial Sequence Synthetic primer
tccaccccccaaaatcaac

SEQ ID NO: 111 - DNA, 21 bp, Artificial Sequence
  Description of Artificial Sequence Synthetic primer
caaagtgcccttctgctcctt

SEQ ID NO: 112 - DNA, 24 bp, Artificial Sequence
  Description of Artificial Sequence Synthetic primer
cacaacgatcttctacacgtgaca

SEQ ID NO: 113 - DNA, 28 bp, Artificial Sequence
  Description of Artificial Sequence Synthetic primer
agttaaacagactggaaaacatggtaaa

SEQ ID NO: 114 - DNA, 25 bp, Artificial Sequence
  Description of Artificial Sequence Synthetic primer
cctttgtttgttaactgctctttcc

SEQ ID NO: 115 - DNA, 25 bp, Artificial Sequence
  Description of Artificial Sequence Synthetic primer
ccaaagaacagacatgcagttattg

SEQ ID NO: 116 - DNA, 19 bp, Artificial Sequence
  Description of Artificial Sequence Synthetic primer
accttggcctctccaagca

SEQ ID NO: 117 - DNA, 21 bp, Artificial Sequence
  Description of Artificial Sequence Synthetic primer
caggccacactccacttttgt

SEQ ID NO: 118 - DNA, 26 bp, Artificial Sequence
  Description of Artificial Sequence Synthetic primer
gcatatgaccacagtatcacaatcaa

SEQ ID NO: 119 - DNA, 23 bp, Artificial Sequence
  Description of Artificial Sequence Synthetic primer
tcacattttttgttgcagtccaa

SEQ ID NO: 120 - DNA, 21 bp, Artificial Sequence
  Description of Artificial Sequence Synthetic primer
gttttacacccagcgatgctt

SEQ ID NO: 121 - DNA, 20 bp, Artificial Sequence
  Description of Artificial Sequence Synthetic primer
ctgcccacagcctctttttc

SEQ ID NO: 122 - DNA, 23 bp, Artificial Sequence
  Description of Artificial Sequence Synthetic primer
aataacctgcattcaccgaagag

SEQ ID NO: 123 - DNA, 19 bp, Artificial Sequence
  Description of Artificial Sequence Synthetic primer
ccgctacacgttggtgcta

SEQ ID NO: 124 - DNA, 23 bp, Artificial Sequence
  Description of Artificial Sequence Synthetic primer
aggaatctccgagttgaggaaaa

SEQ ID NO: 125 - DNA, 26 bp, Artificial Sequence
  Description of Artificial Sequence Synthetic primer
acaagtttaatgcaacaggtgacaac

SEQ ID NO: 126 - DNA, 25 bp, Artificial Sequence
  Description of Artificial Sequence Synthetic primer
acaatttggcatttgagccttttcc

SEQ ID NO: 127 - DNA, 25 bp, Artificial Sequence
  Description of Artificial Sequence Synthetic primer
aacactggcttataaagtccatggt

SEQ ID NO: 128 - DNA, 21 bp, Artificial Sequence
  Description of Artificial Sequence Synthetic primer
caagtgggtgtgagcagcttt

SEQ ID NO: 129 - DNA, 30 bp, Artificial Sequence
  Description of Artificial Sequence Synthetic primer
aatatctttaaataacacaactcccagaca

SEQ ID NO: 130 - DNA, 20 bp, Artificial Sequence
  Description of Artificial Sequence Synthetic primer
gcagatgccctccaagatgt

SEQ ID NO: 131 - DNA, 20 bp, Artificial Sequence
  Description of Artificial Sequence Synthetic primer
aggcccctttccttctgaaa

SEQ ID NO: 132 - DNA, 18 bp, Artificial Sequence
  Description of Artificial Sequence Synthetic primer
gccatgcagggcctagct

SEQ ID NO: 133 - DNA, 24 bp, Artificial Sequence
  Description of Artificial Sequence Synthetic primer
tggcttttattagcgattcatgaa

SEQ ID NO: 134 - DNA, 20 bp, Artificial Sequence
  Description of Artificial Sequence Synthetic primer
agggaagctgccacaagtga

SEQ ID NO: 135 - DNA, 27 bp, Artificial Sequence
  Description of Artificial Sequence Synthetic primer
ttttaagtaccacttttcctccaacaa

SEQ ID NO: 136 - DNA, 26 bp, Artificial Sequence
  Description of Artificial Sequence Synthetic primer
agtgtaagttcagtctgatggaaacc

SEQ ID NO: 137 - DNA, 25 bp, Artificial Sequence
  Description of Artificial Sequence Synthetic primer
ctcaggactctctgctagtacaagt

SEQ ID NO: 138 - DNA, 18 bp, Artificial Sequence
  Description of Artificial Sequence Synthetic primer
tggcgtccaggtccttga

SEQ ID NO: 139 - DNA, 20 bp, Artificial Sequence
  Description of Artificial Sequence Synthetic primer
gcccaaaggcgtagaaggtt

SEQ ID NO: 140 - DNA, 26 bp, Artificial Sequence
  Description of Artificial Sequence Synthetic primer
ggattaagtagagttttgccaaaagc

SEQ ID NO: 141 - DNA, 20 bp, Artificial Sequence
  Description of Artificial Sequence Synthetic primer
gggcccaaaatagggagtgt

SEQ ID NO: 142 - DNA, 23 bp, Artificial Sequence
  Description of Artificial Sequence Synthetic primer
ccccatgattgtaagttcttcca

SEQ ID NO: 143 - DNA, 21 bp, Artificial Sequence
  Description of Artificial Sequence Synthetic primer
cattcagcagatgggcagact

SEQ ID NO: 144 - DNA, 28 bp, Artificial Sequence
  Description of Artificial Sequence Synthetic primer
tgccttaactagctcaatttatcttgtg

SEQ ID NO: 145 - DNA, 19 bp, Artificial Sequence
  Description of Artificial Sequence Synthetic primer
tgctgcaaactgggatcca

SEQ ID NO: 146 - DNA, 22 bp, Artificial Sequence
  Description of Artificial Sequence Synthetic primer
ccacagttttggcagtgaacaa

SEQ ID NO: 147 - DNA, 27 bp, Artificial Sequence
  Description of Artificial Sequence Synthetic primer
caagatggatccactactttacatgga

SEQ ID NO: 148 - DNA, 27 bp, Artificial Sequence
  Description of Artificial Sequence Synthetic primer
ttaggatgagtgtgaaatcaaatacga

SEQ ID NO: 149 - DNA, 19 bp, Artificial Sequence
  Description of Artificial Sequence Synthetic primer
ggctccgaaatggcatctc

SEQ ID NO: 150 - DNA, 30 bp, Artificial Sequence
  Description of Artificial Sequence Synthetic primer
gtgcaaattgacttttacattcaactttag

SEQ ID NO: 151 - DNA, 23 bp, Artificial Sequence
  Description of Artificial Sequence Synthetic primer
tcacacccccatactcttctgtt

SEQ ID NO: 152 - DNA, 25 bp, Artificial Sequence
  Description of Artificial Sequence Synthetic primer
cccataaagcaattcacggatacag

SEQ ID NO: 153 - DNA, 19 bp, Artificial Sequence
  Description of Artificial Sequence Synthetic primer
gcttgggaaaccgcacttt

SEQ ID NO: 154 - DNA, 20 bp, Artificial Sequence
  Description of Artificial Sequence Synthetic primer
atgcatgggccattgatctt

SEQ ID NO: 155 - DNA, 29 bp, Artificial Sequence
  Description of Artificial Sequence Synthetic primer
ggtcacataaaaatacatgaggatgataa

SEQ ID NO: 156 - DNA, 21 bp, Artificial Sequence
  Description of Artificial Sequence Synthetic primer
tgactggccctgcagaatact

SEQ ID NO: 157 - DNA, 18 bp, Artificial Sequence
  Description of Artificial Sequence Synthetic primer
cccactccccatcaacca

SEQ ID NO: 158 - DNA, 24 bp, Artificial Sequence
  Description of Artificial Sequence Synthetic primer
aaatggacagacacatgctgaact

SEQ ID NO: 159 - DNA, 24 bp, Artificial Sequence
  Description of Artificial Sequence Synthetic primer
ccaagagagaccagtgctcaaata

SEQ ID NO: 160 - DNA, 23 bp, Artificial Sequence
  Description of Artificial Sequence Synthetic primer
ttggcaaacggatgagttaaaaa

SEQ ID NO: 161 - DNA, 26 bp, Artificial Sequence
  Description of Artificial Sequence Synthetic primer
tgaatgaagatatgaaagctgggctt

SEQ ID NO: 162 - DNA, 28 bp, Artificial Sequence
  Description of Artificial Sequence Synthetic primer
atgaatacacagtctggtagagtcttct

SEQ ID NO: 163 - DNA, 22 bp, Artificial Sequence
  Description of Artificial Sequence Synthetic primer
gatccaacgtgcagaagcctat

SEQ ID NO: 164 - DNA, 22 bp, Artificial Sequence
  Description of Artificial Sequence Synthetic primer
gggctttattattgggcaaaca

SEQ ID NO: 165 - DNA, 21 bp, Artificial Sequence
  Description of Artificial Sequence Synthetic primer
gcatggcaggtagtgaggaaa

SEQ ID NO: 166 - DNA, 26 bp, Artificial Sequence
  Description of Artificial Sequence Synthetic primer
accctctggctatactaacaccaact

SEQ ID NO: 167 - DNA, 30 bp, Artificial Sequence
  Description of Artificial Sequence Synthetic primer
gcttctctttattttcaacagtttctttac

SEQ ID NO: 168 - DNA, 20 bp, Artificial Sequence
  Description of Artificial Sequence Synthetic primer
cctctccagcccacagtgat

SEQ ID NO: 169 - DNA, 27 bp, Artificial Sequence
  Description of Artificial Sequence Synthetic primer
gagctgagggcctaagataaataaagt

SEQ ID NO: 170 - DNA, 19 bp, Artificial Sequence
  Description of Artificial Sequence Synthetic primer
gctgcttgcgcctcttttt

SEQ ID NO: 171 - DNA, 23 bp, Artificial Sequence
  Description of Artificial Sequence Synthetic primer
tccaaagcagcttaggtgaaaaa

SEQ ID NO: 172 - DNA, 24 bp, Artificial Sequence
  Description of Artificial Sequence Synthetic primer
catgtcgcagggttaagtatgatg

SEQ ID NO: 173 - DNA, 25 bp, Artificial Sequence
  Description of Artificial Sequence Synthetic primer
ggcaaagagagacatttcactcaga

SEQ ID NO: 174 - DNA, 20 bp, Artificial Sequence
  Description of Artificial Sequence Synthetic primer
attccaaggcccccttaaga

SEQ ID NO: 175 - DNA, 20 bp, Artificial Sequence
  Description of Artificial Sequence Synthetic probe
aacggactttagaatcttct

SEQ ID NO: 176 - DNA, 15 bp, Artificial Sequence
  Description of Artificial Sequence Synthetic probe
aggcctggacaagga

SEQ ID NO: 177 - DNA, 20 bp, Artificial Sequence
  Description of Artificial Sequence Synthetic probe
agtttattcatggagcatgc

SEQ ID NO: 178 - DNA, 16 bp, Artificial Sequence
  Description of Artificial Sequence Synthetic probe
acattgtgcaggaggg

SEQ ID NO: 179 - DNA, 17 bp, Artificial Sequence
  Description of Artificial Sequence Synthetic probe
ctgagcttaggatcatc

SEQ ID NO: 180 - DNA, 16 bp, Artificial Sequence
  Description of Artificial Sequence Synthetic probe
catcaggccgcagtcc

SEQ ID NO: 181 - DNA, 17 bp, Artificial Sequence
  Description of Artificial Sequence Synthetic probe
ctgactcccagttattt

SEQ ID NO: 182 - DNA, 16 bp, Artificial Sequence
  Description of Artificial Sequence Synthetic probe
ttgcccagcctctttg

SEQ ID NO: 183 - DNA, 16 bp, Artificial Sequence
  Description of Artificial Sequence Synthetic probe
ttttcaagcacaaccc

SEQ ID NO: 184 - DNA, 15 bp, Artificial Sequence
  Description of Artificial Sequence Synthetic probe
ccggatcgccatcag

SEQ ID NO: 185 - DNA, 20 bp, Artificial Sequence
  Description of Artificial Sequence Synthetic probe
ttccaagatcatagacttac

SEQ ID NO: 186 - DNA, 20 bp, Artificial Sequence
  Description of Artificial Sequence Synthetic probe
actttgtaaagcaaataatg

SEQ ID NO: 187 - DNA, 16 bp, Artificial Sequence
  Description of Artificial Sequence Synthetic probe
ccttcagggtgttcgg

SEQ ID NO: 188 - DNA, 18 bp, Artificial Sequence
  Description of Artificial Sequence Synthetic probe
aaagaagtccgagatatt

SEQ ID NO: 189 - DNA, 16 bp, Artificial Sequence
  Description of Artificial Sequence Synthetic probe
aacttgcctagaactc

SEQ ID NO: 190 - DNA, 14 bp, Artificial Sequence
  Description of Artificial Sequence Synthetic probe
tttcaccaaaaccc

SEQ ID NO: 191 - DNA, 17 bp, Artificial Sequence
  Description of Artificial Sequence Synthetic probe
ccacaagactggcagag

SEQ ID NO: 192 - DNA, 20 bp, Artificial Sequence
  Description of Artificial Sequence Synthetic probe
tggaaacagtttggattgta

SEQ ID NO: 193 - DNA, 17 bp, Artificial Sequence
  Description of Artificial Sequence Synthetic probe
ttgtgccccacactaac

SEQ ID NO: 194 - DNA, 18 bp, Artificial Sequence
  Description of Artificial Sequence Synthetic probe
aaatgtacggagcttcat

SEQ ID NO: 195 - DNA, 16 bp, Artificial Sequence
  Description of Artificial Sequence Synthetic probe
tcagcatcacttcagc

SEQ ID NO: 196 - DNA, 15 bp, Artificial Sequence
  Description of Artificial Sequence Synthetic probe
atgcctgcctttcaa

SEQ ID NO: 197 - DNA, 18 bp, Artificial Sequence
  Description of Artificial Sequence Synthetic probe
tgtgaggtttgtttgtcc

SEQ ID NO: 198 - DNA, 17 bp, Artificial Sequence
  Description of Artificial Sequence Synthetic probe
catgagagcccagaaca

SEQ ID NO: 199 - DNA, 20 bp, Artificial Sequence
  Description of Artificial Sequence Synthetic probe
cctacaggatacacgtgaga

SEQ ID NO: 200 - DNA, 19 bp, Artificial Sequence
  Description of Artificial Sequence Synthetic probe
catttttagctcgctcatt

SEQ ID NO: 201 - DNA, 17 bp, Artificial Sequence
  Description of Artificial Sequence Synthetic probe
aggctagaggctgaggg

SEQ ID NO: 202 - DNA, 14 bp, Artificial Sequence
  Description of Artificial Sequence Synthetic probe
atcatgccaattcc

SEQ ID NO: 203 - DNA, 15 bp, Artificial Sequence
  Description of Artificial Sequence Synthetic probe
catacctgtaatccc

SEQ ID NO: 204 - DNA, 17 bp, Artificial Sequence
  Description of Artificial Sequence Synthetic probe
tatggatgccgtgggag

SEQ ID NO: 205 - DNA, 23 bp, Artificial Sequence
  Description of Artificial Sequence Synthetic probe
ttgagtgattgttaatgttgtct

SEQ ID NO: 206 - DNA, 17 bp, Artificial Sequence
  Description of Artificial Sequence Synthetic probe
agccactaaccaactag

SEQ ID NO: 207 - DNA, 15 bp, Artificial Sequence
  Description of Artificial Sequence Synthetic probe
ctctctgccatcccc

SEQ ID NO: 208 - DNA, 14 bp, Artificial Sequence
  Description of Artificial Sequence Synthetic probe
ctggagcaggtggc

SEQ ID NO: 209 - DNA, 18 bp, Artificial Sequence
  Description of Artificial Sequence Synthetic probe
tgagttttaagagatccc

SEQ ID NO: 210 - DNA, 17 bp, Artificial Sequence
  Description of Artificial Sequence Synthetic probe
ttcacgcactgtccctc

SEQ ID NO: 211 - DNA, 18 bp, Artificial Sequence
  Description of Artificial Sequence Synthetic probe
aaactgaatggcacgaaa

SEQ ID NO: 212 - DNA, 17 bp, Artificial Sequence
  Description of Artificial Sequence Synthetic probe
atgctccggaaggctca

SEQ ID NO: 213 - DNA, 21 bp, Artificial Sequence
  Description of Artificial Sequence Synthetic probe
cagtgtagagctcttgtttta

SEQ ID NO: 214 - DNA, 20 bp, Artificial Sequence
  Description of Artificial Sequence Synthetic probe
acttttcaaggcttatattc

SEQ ID NO: 215 - DNA, 20 bp, Artificial Sequence
  Description of Artificial Sequence Synthetic probe
ctgcatattgttccagataa

SEQ ID NO: 216 - DNA, 13 bp, Artificial Sequence
  Description of Artificial Sequence Synthetic probe
ccccccaaatatt

SEQ ID NO: 217 - DNA, 16 bp, Artificial Sequence
  Description of Artificial Sequence Synthetic probe
tgattagacaaggccc

SEQ ID NO: 218 - DNA, 15 bp, Artificial Sequence
  Description of Artificial Sequence Synthetic probe
agagacacagccctc

SEQ ID NO: 219 - DNA, 17 bp, Artificial Sequence
  Description of Artificial Sequence Synthetic probe
agcactttcccttggtg

SEQ ID NO: 220 - DNA, 18 bp, Artificial Sequence
  Description of Artificial Sequence Synthetic probe
taggctggatgctaccca

SEQ ID NO: 221 - DNA, 22 bp, Artificial Sequence
  Description of Artificial Sequence Synthetic probe
ctagtgtcttttttttcttcac

SEQ ID NO: 222 - DNA, 22 bp, Artificial Sequence
  Description of Artificial Sequence Synthetic probe
acttttctgaattgctatgact

SEQ ID NO: 223 - DNA, 20 bp, Artificial Sequence
  Description of Artificial Sequence Synthetic probe
catcaaggatacaaatctac

SEQ ID NO: 224 - DNA, 16 bp, Artificial Sequence
  Description of Artificial Sequence Synthetic probe
cccgctcctgcaggag

SEQ ID NO: 225 - DNA, 14 bp, Artificial Sequence
  Description of Artificial Sequence Synthetic probe
ccgtggataaattg

SEQ ID NO: 226 - DNA, 17 bp, Artificial Sequence
  Description of Artificial Sequence Synthetic probe
acagctatctgctggct

SEQ ID NO: 227 - DNA, 19 bp, Artificial Sequence
  Description of Artificial Sequence Synthetic probe
ccaaagagtgatagtcttt

SEQ ID NO: 228 - DNA, 16 bp, Artificial Sequence
  Description of Artificial Sequence Synthetic probe
tccaccctcatcaccc

SEQ ID NO: 229 - DNA, 21 bp, Artificial Sequence
  Description of Artificial Sequence Synthetic probe
aaatgatagttcgactcgtct

SEQ ID NO: 230 - DNA, 16 bp, Artificial Sequence
  Description of Artificial Sequence Synthetic probe
ctccacactcttgggc

SEQ ID NO: 231 - DNA, 20 bp, Artificial Sequence
  Description of Artificial Sequence Synthetic probe
tagaatggttgagtgcaaat

SEQ ID NO: 232 - DNA, 18 bp, Artificial Sequence
  Description of Artificial Sequence Synthetic probe
atggcagatctgataccc

SEQ ID NO: 233 - DNA, 14 bp, Artificial Sequence
  Description of Artificial Sequence Synthetic probe
ccagaggaatcccc

SEQ ID NO: 234 - DNA, 15 bp, Artificial Sequence
  Description of Artificial Sequence Synthetic probe
ctgcagcaaacccca

SEQ ID NO: 235 - DNA, 19 bp, Artificial Sequence
  Description of Artificial Sequence Synthetic probe
tgtcagctcaaaaaccaga

SEQ ID NO: 236 - DNA, 14 bp, Artificial Sequence
  Description of Artificial Sequence Synthetic probe
agggagagaaaacc

SEQ ID NO: 237 - DNA, 17 bp, Artificial Sequence
  Description of Artificial Sequence Synthetic probe
actgagtgccttcattt

SEQ ID NO: 238 - DNA, 17 bp, Artificial Sequence
  Description of Artificial Sequence Synthetic probe
ctgcagatgtagttgcc

SEQ ID NO: 239 - DNA, 23 bp, Artificial Sequence
  Description of Artificial Sequence Synthetic probe
ttcacagtaaacctaagaacact

SEQ ID NO: 240 - DNA, 16 bp, Artificial Sequence
  Description of Artificial Sequence Synthetic probe
actgcaggaccagaag

SEQ ID NO: 241 - DNA, 15 bp, Artificial Sequence
  Description of Artificial Sequence Synthetic probe
cctccgtaggcatca

SEQ ID NO: 242 - DNA, 18 bp, Artificial Sequence
  Description of Artificial Sequence Synthetic probe
tgcaacactgtgtattag

SEQ ID NO: 243 - DNA, 19 bp, Artificial Sequence
  Description of Artificial Sequence Synthetic probe
ttgcttagacattgttttc

SEQ ID NO: 244 - DNA, 17 bp, Artificial Sequence
  Description of Artificial Sequence Synthetic probe
caaggaagggtgctgca

SEQ ID NO: 245 - DNA, 19 bp, Artificial Sequence
  Description of Artificial Sequence Synthetic probe
tccttaatgtcacaatgtt

SEQ ID NO: 246 - DNA, 18 bp, Artificial Sequence
  Description of Artificial Sequence Synthetic probe
caaatggtagctgaaaaa

SEQ ID NO: 247 - DNA, 14 bp, Artificial Sequence
  Description of Artificial Sequence Synthetic probe
tggaagcagaaggc

SEQ ID NO: 248 - DNA, 19 bp, Artificial Sequence
  Description of Artificial Sequence Synthetic probe
cctctgaaacacattcttg

SEQ ID NO: 249 - DNA, 24 bp, Artificial Sequence
  Description of Artificial Sequence Synthetic probe
ttctatgtagtttggtaattatca

SEQ ID NO: 250 - DNA, 14 bp, Artificial Sequence
  Description of Artificial Sequence Synthetic probe
agtgccaataatcg

SEQ ID NO: 251 - DNA, 15 bp, Artificial Sequence
  Description of Artificial Sequence Synthetic probe
agtgctccaaatgtc

SEQ ID NO: 252 - DNA, 18 bp, Artificial Sequence
  Description of Artificial Sequence Synthetic probe
aaaggaaccagtcagctg

SEQ ID NO: 253 - DNA, 17 bp, Artificial Sequence
  Description of Artificial Sequence Synthetic probe
cagtgttatgcactttc

SEQ ID NO: 254 - DNA, 18 bp, Artificial Sequence
  Description of Artificial Sequence Synthetic probe
aatccctgtgtagactgt

SEQ ID NO: 255 - DNA, 16 bp, Artificial Sequence
  Description of Artificial Sequence Synthetic probe
ctgtcttgtaaaagcc

SEQ ID NO: 256 - DNA, 17 bp, Artificial Sequence
  Description of Artificial Sequence Synthetic probe
agtctcgaacagcggtt

SEQ ID NO: 257 - DNA, 16 bp, Artificial Sequence
  Description of Artificial Sequence Synthetic probe
tgctgtgccagtgtga

SEQ ID NO: 258 - DNA, 18 bp, Artificial Sequence
  Description of Artificial Sequence Synthetic probe
ctggtgaatgtaaacaat

SEQ ID NO: 259 - DNA, 20 bp, Artificial Sequence
  Description of Artificial Sequence Synthetic probe
ttcaaacagactttaacctc

SEQ ID NO: 260 - DNA, 14 bp, Artificial Sequence
  Description of Artificial Sequence Synthetic probe
cccccagactttgg

SEQ ID NO: 261 - DNA, 14 bp, Artificial Sequence
  Description of Artificial Sequence Synthetic probe
ctctcccaattttc